(19) United States
(12) Patent Application Publication
(10) Pub. No.: US 2026/0301854 A1
(43) Pub. Date: Oct. 1, 2026

(54) DNA PROGRAMMING LANGUAGE AND SIMULATOR USING RECOGNITION SCIENCE-DERIVED STRUCTURAL AND COHERENCE CONSTRAINTS

(71) Applicant: Jonathan Washburn, Austin, TX (US)

(72) Inventor: Jonathan Washburn, Austin, TX (US)

(21) Appl. No.: 19/629,680

(22) Filed: Mar. 26, 2026

Related U.S. Application Data
(60) Provisional application No. 63/778,862, filed on Mar. 27, 2025.

(51) Int. Cl. G16B 15/00; G16B 30/00

(52) U.S. Cl. CPC G16B 15/00; G16B 30/00

(57) ABSTRACT

A formal DNA programming language, simulation system, and computer-implemented method are disclosed for evaluating candidate DNA designs. A candidate DNA design is represented as a tuple D=(S, H, E), where S is a sequence component, His a shape component, and E is an energy component. The sequence component is used to compute a sequence stability score, the shape component encodes helical pitch and groove ratio, and the energy component encodes a coherence-related state and an associated rate. A system receives the candidate DNA design, validates sequence and geometric constraints, performs structural stability evaluation and coherence validation, computes a predicted functional output, and outputs a validity result and the predicted functional output. The disclosed framework may be used for therapeutic construct design, regulatory element tuning, and DNA computing applications.

### CROSS-REFERENCE TO RELATED APPLICATIONS

This application claims benefit of priority to U.S. Provisional Patent Application No. 63/778,862, titled “A Formal Language and System for Programming DNA Based on Recognition Physics Principles” filed on Mar. 27, 2025.

### BACKGROUND

The present invention relates generally to computational molecular biology, synthetic biology, and formal language systems for biomolecular design. More particularly, the invention relates to computer-implemented design, representation, simulation, and evaluation of candidate DNA molecules using a formal DNA programming language that jointly encodes sequence information, helical geometry, and coherence-related energetic state. Conventional DNA design tools typically address only selected aspects of DNA behavior, such as sequence composition, thermodynamic stability, secondary structure, hybridization propensity, codon usage, or empirical expression prediction. Although such tools may be useful within their respective domains, they generally do not provide a single executable representation by which a candidate DNA design can be defined, validated, and computationally evaluated across sequence-level, geometry-level, and coherence-related constraints within a unified framework.

Existing approaches also tend to rely heavily on empirically fitted models, heuristic scoring systems, or isolated simulation domains that are not integrated into a common formal language for DNA programs. As a result, conventional systems generally do not provide a deterministic workflow in which a candidate DNA design is represented as a structured program object, screened for sequence admissibility, evaluated for helical pitch and groove-ratio compatibility, tested for structural stability, validated against a discrete coherence-state framework, and used to generate a predicted functional output from the same underlying representation. In particular, existing DNA design tools do not generally incorporate a unified formalism capable of simultaneously encoding sequence, shape, and coherence-related state, nor do they provide a first-principles output-prediction framework tied to that same representation. Accordingly, a need exists for a formal DNA programming language, simulation system, and computer-implemented evaluation method that enables candidate DNA molecules to be represented and analyzed within a single coherent computational framework.

### SUMMARY

The present invention provides a formal DNA programming language, a simulation system, and a computer-implemented method for representing, evaluating, selecting, and refining candidate DNA designs using a unified sequence-shape-energy framework. In one aspect, a candidate DNA design is represented as a DNARP program having a sequence component, a shape component, and an energy component. The sequence component captures ordered sequence content and may be used to compute a sequence stability score. The shape component captures helical geometry, including helical pitch and groove ratio. The energy component captures a coherence-related state and an associated rate. By representing candidate DNA designs in this unified form, the disclosed framework allows a candidate to be processed as a machine-usable program object rather than as an isolated biological sequence or an informal collection of parameters.

In another aspect, the invention provides a deterministic computational workflow for evaluating a candidate DNARP program. In one embodiment, a system receives a candidate DNA program, parses the candidate into sequence, shape, and energy components, computes a sequence stability score from the sequence component, determines whether the shape component satisfies geometric admissibility constraints, performs a structural stability evaluation using a DNA Lagrangian, performs a coherence validation using a DNA recognition operator, computes a predicted functional output using a recognition transform, and outputs a result indicating whether the candidate is valid and, if valid, a corresponding predicted output. In this manner, the invention provides a unified evaluation framework in which sequence-level admissibility, geometry-level admissibility, structural stability, coherence validity, and output prediction are all determined from the same formal representation.

In one embodiment, the sequence component is evaluated according to weighted complementary contributions, such that CG-supported contributions and AT-supported contributions are distinguished in computing a sequence stability score. In one embodiment, the shape component is represented as H=(P, G), where P is helical pitch and G is groove ratio, and the candidate is evaluated against permitted geometric ranges. In one embodiment, the energy component is represented as E=(E_n, R), where E_n is a discrete coherence-related energy level and R is an associated output rate. In one expressly described implementation, the coherence-related energy level satisfies E_n=n*E_coh, the associated rate satisfies R=n*R_0, and n is selected from {1, 2, 3}. These relationships provide a structured way to encode and evaluate candidate DNA designs using discrete and machine-testable criteria.

In one embodiment, the structural stability evaluation is based on a DNA Lagrangian that receives at least a helical pitch value associated with the candidate design and produces or approximates a structural profile used to determine whether the candidate remains within a prescribed stability tolerance band. In one embodiment, coherence validation is based on a DNA-adapted recognition operator used to determine whether a requested coherence-related state is an allowed and admissible state for the candidate configuration. In one embodiment, output prediction is based on a recognition transform that combines a sequence-derived stability term, a coherence-related term, and a geometry-related term to generate a predicted functional output for the candidate DNA program. The system may therefore accept or reject a candidate according to objective thresholds and validation criteria and may further use the resulting output to compare, rank, or refine candidate designs.

In one embodiment, the invention is implemented as a processor-based system including one or more processors, memory, storage, and one or more interfaces configured to receive candidate DNA programs and output candidate evaluation results. In one embodiment, the system includes an input validation module, a stability analysis module, a coherence validation module, a function prediction module, and an output module. In one embodiment, the invention is implemented as executable instructions stored on a non-transitory computer-readable medium and configured to cause one or more processors to perform the disclosed candidate-receipt, parsing, validation, stability-analysis, coherence-validation, and output-prediction operations. In one embodiment, the invention is implemented in a local computing environment, a remote computing environment, or a cloud-based service.

In one embodiment, the disclosed framework is used in a therapeutic design workflow in which a therapeutic target is identified, a candidate therapeutic DNA construct is represented as a DNARP program, and the candidate therapeutic DNA construct is evaluated using the disclosed sequence, geometry, structural stability, coherence, and output stages to determine whether the construct is suitable for the selected therapeutic objective. In another embodiment, the disclosed framework is used in a regulatory element tuning workflow in which a candidate promoter or regulatory element and one or more sequence variants are evaluated against a target output criterion, iteratively refined, and used to select an optimized regulatory element. In another embodiment, the disclosed framework is used in a DNA computing workflow in which a toehold switch or DNA logic construct is evaluated in view of a trigger input sequence, a gate sequence region, an OFF state, and an ON state so that switching behavior may be assessed under the same sequence-shape-energy framework.

Accordingly, the invention provides a formal DNA design language, a simulator architecture, and a computer-implemented validation-and-prediction workflow that together enable candidate DNA molecules to be represented, screened, validated, compared, and refined within a single coherent computational framework. This unified approach improves determinacy and technical integration relative to systems that separately handle sequence analysis, geometry evaluation, and functional prediction, and supports applications including therapeutic construct design, regulatory element engineering, and DNA-based computing.

In one implementation, the disclosed framework provides a technical improvement in DNA design computing by converting candidate molecules into a unified, machine-usable DNARP tuple representation D=(S,H,E) that is processed through deterministic, staged validation gates. Unlike heuristic or empirically fitted pipelines that treat sequence scoring, geometric screening, stability analysis, and functional prediction as separate tools or loosely coupled steps, the disclosed system applies a single formal representation across sequence admissibility, geometry admissibility, structural stability evaluation, coherence-state validation, and output prediction.

This deterministic multi-stage structure enables repeatable machine screening, objective pass/fail outcomes under disclosed criteria, and consistent comparison or ranking of candidates based on a common computational basis rather than ad hoc scoring across incompatible domains.

### BRIEF DESCRIPTION OF THE DRAWINGS

FIG. 1 is a block diagram illustrating a DNARP design and simulation system according to one embodiment of the invention.

FIG. 2 is a diagram illustrating a DNARP program tuple having a sequence component, a shape component, and an energy component according to one embodiment of the invention.

FIG. 3 is a flow diagram illustrating a DNARP evaluation method for receiving, parsing, validating, and evaluating a candidate DNA program according to one embodiment of the invention.

FIG. 4 is a diagram illustrating a sequence evaluation subsystem for determining a sequence stability score from candidate sequence content according to one embodiment of the invention.

FIG. 5 is a diagram illustrating a shape evaluation subsystem for evaluating helical pitch and groove ratio of a candidate DNA design according to one embodiment of the invention.

FIG. 6 is a diagram illustrating structural stability evaluation of a candidate DNA design using a DNA Lagrangian and a resulting stability profile according to one embodiment of the invention.

FIG. 7 is a diagram illustrating coherence validation of a candidate DNA design using a DNA recognition operator and an allowed coherence-state framework according to one embodiment of the invention.

FIG. 8 is a diagram illustrating output prediction for a candidate DNA design using a recognition transform and a predicted functional output according to one embodiment of the invention.

FIG. 9 is a workflow diagram illustrating therapeutic design of a candidate therapeutic DNA construct for a selected therapeutic target according to one embodiment of the invention.

FIG. 10 is a workflow diagram illustrating regulatory element tuning for a candidate promoter or regulatory element according to one embodiment of the invention.

FIG. 11 is a workflow diagram illustrating DNA computing using a toehold switch or DNA logic construct, a trigger input sequence, and OFF-state and ON-state operation according to one embodiment of the invention.

FIG. 12 is a block diagram illustrating a computing environment configured to implement the disclosed DNARP design and simulation system according to one embodiment of the invention.

### DETAILED DESCRIPTION

Numerical values disclosed herein may be used as exact values, approximate values, bounded ranges, threshold values, tolerance-based values, or implementation-specific values derived from the disclosed framework, depending on the needs of a particular embodiment. Where a range is disclosed, the range includes values within the stated interval as well as implementations centered on preferred values, constrained by tolerance bands, or evaluated according to pass/fail criteria derived from the disclosed methods.

As used herein, a DNA molecule may include, without limitation where context permits, a full-length DNA molecule, a DNA fragment, a duplex, a coding region, a non-coding region, a regulatory element, a promoter, a therapeutic construct, a design candidate, or another DNA-containing construct represented and evaluated under the disclosed formal framework. A candidate DNA program, candidate DNA design, candidate construct, candidate sequence, or candidate molecule may be used interchangeably where the context indicates that the subject is a design instance represented in the disclosed formal language and processed by the disclosed computational workflow. Likewise, the terms valid, invalid, accepted, rejected, passed, failed, admissible, and non-admissible may be used to denote outcomes of one or more disclosed validation operations, including sequence-level validation, shape-level validation, structural stability evaluation, coherence validation, or output-prediction processing.

As used herein, “CG pair” refers to a Watson-Crick C·G base pair in a duplex portion of a candidate DNA design (including predicted duplex pairing in a simulated secondary-structure state), and “AT pair” refers to a Watson-Crick A·T base pair in a duplex portion of the candidate DNA design. In embodiments that evaluate a single-stranded candidate prior to duplex prediction, the system first predicts duplex pairing or local pairing propensity for the candidate (e.g., by a pairing map, predicted stem regions, or another duplex/secondary-structure prediction), and then counts C·G and A·T base pairs within the predicted paired regions for purposes of computing a sequence stability score.

As used herein, “functional unit” refers to (i) a codon triplet for coding-region embodiments, (ii) a fixed-length nucleotide window for non-coding embodiments, or (iii) a labeled sequence block (e.g., trigger, gate, spacer, promoter segment, regulatory segment, or other design block) for logic/regulatory embodiments, where the functional unit is the unit on which a sequence threshold criterion is applied. A DNARP program may explicitly label functional units, or the simulator may infer functional units based on an embodiment profile, an application workflow, or a selected evaluation mode. In one embodiment, when a candidate includes multiple functional units, the candidate is rejected if any functional unit fails to satisfy the threshold criterion.

As used herein, “sequence stability score” S_stab is computed as S_stab=1.5*(#CG pairs)+1.0*(#AT pairs) for a selected evaluation region (e.g., a functional unit, a predicted paired region, or the whole candidate), and is compared to a threshold criterion used to determine sequence-level admissibility.

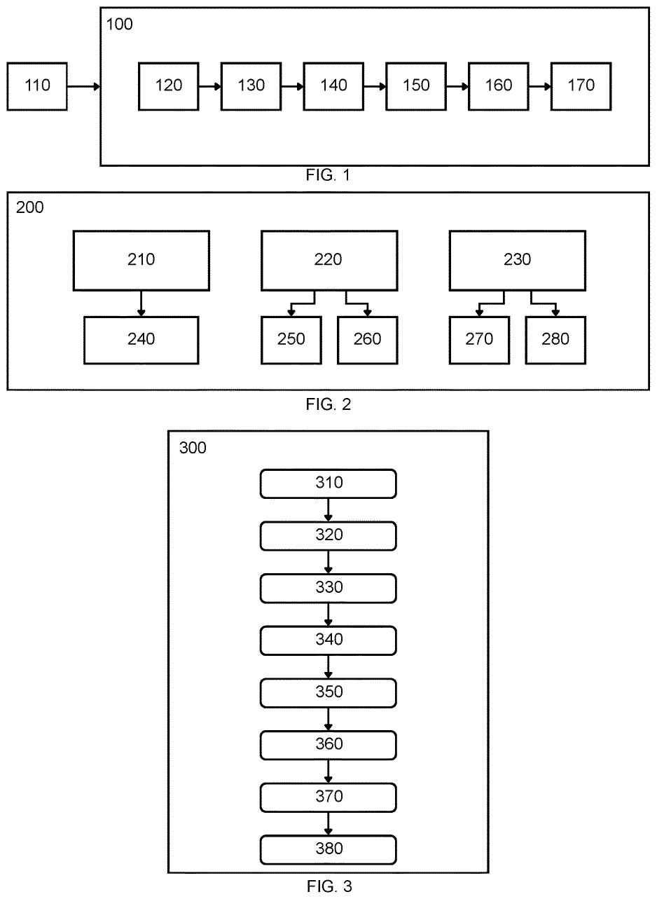

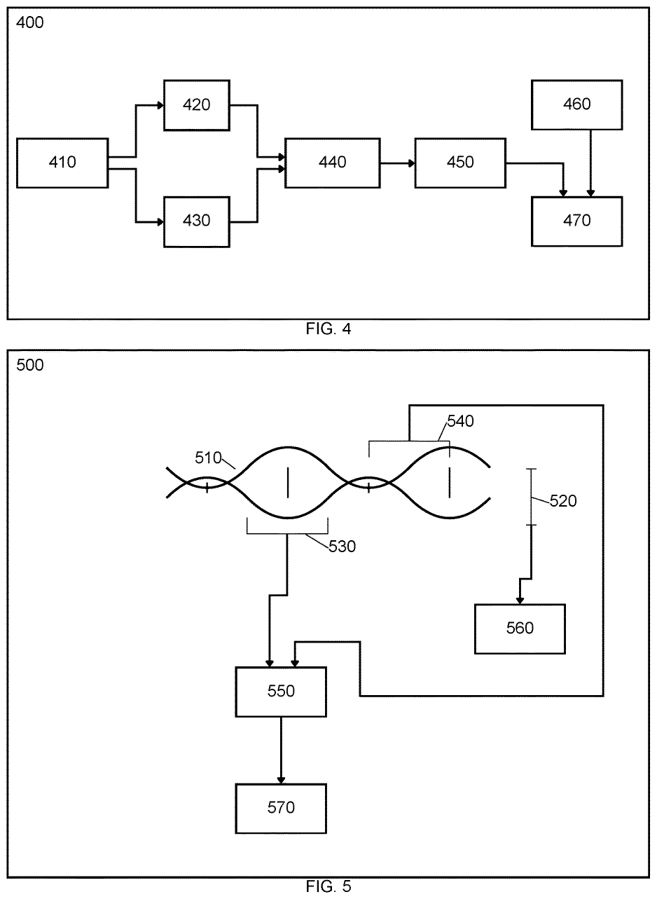

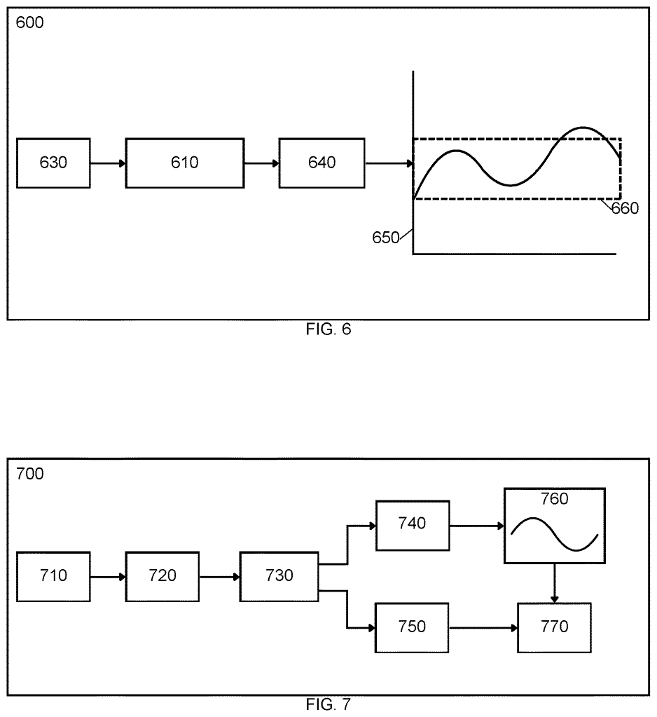

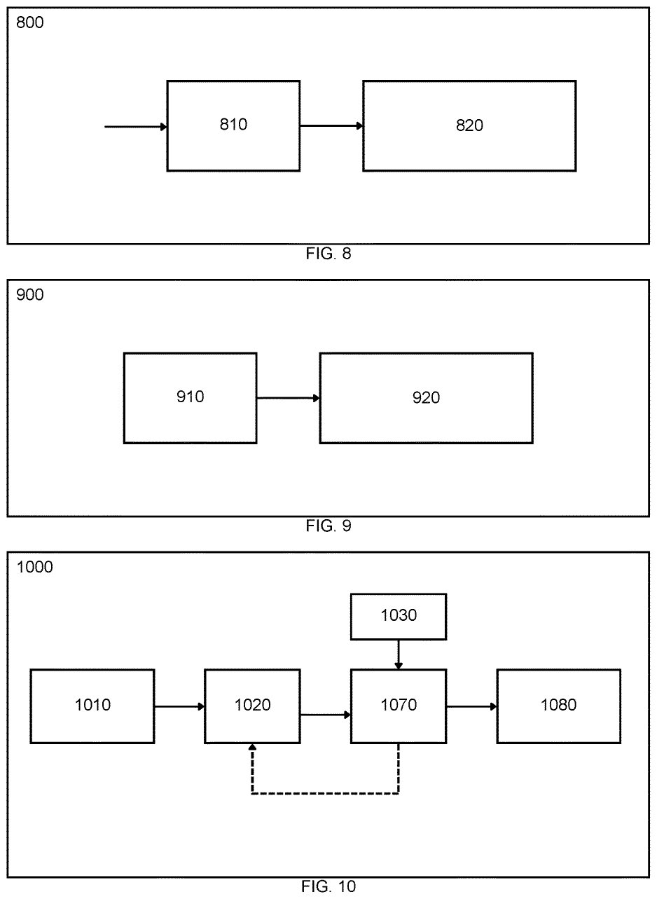

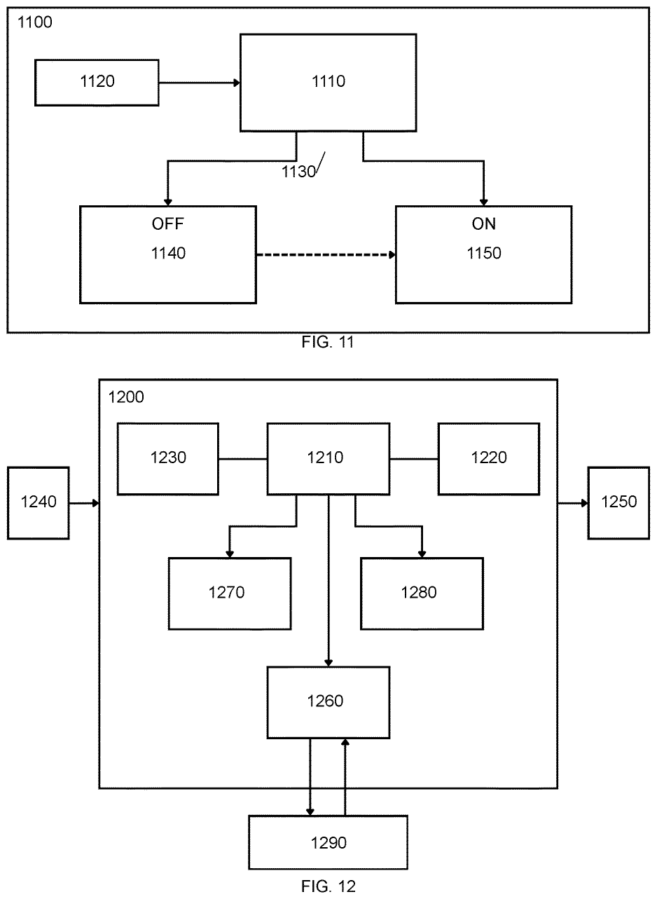

The disclosed invention may be understood with reference to the drawings, each of which is directed to a technical aspect of a common DNA programming and evaluation framework. A high-level DNARP design and simulation system is illustrated in (FIG. 1) as system (100), which may receive a user or API input, define or receive a DNARP program, and process the program through staged computational modules. A DNARP program tuple is illustrated in (FIG. 2) as tuple (200), which provides the formal structure by which a candidate DNA design is represented for subsequent evaluation. A DNARP evaluation method is illustrated in (FIG. 3) as method (300), which shows an ordered computational flow for receiving, parsing, validating, and evaluating a candidate program. Sequence-related processing is further illustrated in (FIG. 4), shape-related processing is further illustrated in (FIG. 5), structural stability evaluation is further illustrated in (FIG. 6), coherence validation is further illustrated in (FIG. 7), and output prediction is further illustrated in (FIG. 8). Application-oriented workflows are illustrated in (FIGS. 9-11), and a generic computing environment suitable for implementing the disclosed system is illustrated in (FIG. 12) as computing environment (1200).

The drawings are provided to illustrate exemplary embodiments and should not be understood as requiring a particular visual arrangement, spatial layout, software architecture, or ordering of operations unless explicitly stated. The same disclosed inventive concepts may be implemented using different internal data structures, interface arrangements, module boundaries, solver routines, or execution environments while remaining within the scope of the present disclosure.

Similarly, where a figure depicts a particular processing stage, module, tuple component, or workflow arrangement, that depiction should be understood as supporting one or more embodiments in which the illustrated operations are performed in the shown order, in a partially reordered sequence, in a combined sequence, in a distributed execution environment, or through equivalent computational structures that implement the same disclosed technical logic.

The drawings are also intended to be read together rather than in isolation. Thus, the high-level system architecture shown in system (100) of (FIG. 1), the tuple-level formal representation shown in tuple (200) of (FIG. 2), the staged evaluation flow shown in method (300) of (FIG. 3), the more detailed analytical portions shown in (FIGS. 4-8), the application workflows shown in (FIGS. 9-11), and the computing support shown in computing environment (1200) of (FIG. 12) collectively describe a unified computer-implemented framework for representing, evaluating, and outputting candidate DNA designs. Unless expressly indicated otherwise, reference numerals in the drawings identify exemplary elements, modules, data representations, processing stages, or computing components that may be used alone or in combination in different embodiments of the disclosed invention.

Recognition Science provides the cost-based and scaling framework from which the DNA-relevant parameters used by the disclosed DNARP system are derived. In the present disclosure, Recognition Science is not introduced as an end in itself, and the present invention does not require restatement or proof of the entire Recognition Science framework. Rather, Recognition Science is used in a constrained and application-specific manner to supply a mathematically grounded basis for defining admissible sequence, shape, and coherence parameters for a candidate DNA design represented and processed by the disclosed DNARP framework. In this sense, Recognition Science serves as the source theory from which the disclosed DNA design language obtains a unified set of governing constants, scaling relations, and validation constructs that are then applied computationally to candidate DNA molecules.

In one aspect, Recognition Science is based on a cost function that measures deviation from an optimal recognition condition. An exemplary form of this cost function is given by:

J(x)=0.5*(x+1/x)−1

This expression is useful because it is symmetric under inversion of x, has a minimum at x=1, and provides a compact measure of recognition overhead or deviation from balanced recognition.

Within the present disclosure, this cost-based foundation is used only to motivate the existence of preferred scales and preferred constrained relationships, rather than to introduce a separate claimed theory of physics. The disclosed DNARP framework uses that cost-minimizing perspective to justify why certain geometric, energetic, and stability-related DNA parameters are treated as preferred or admissible in the computational design process.

Recognition Science further employs a self-similar scaling relation based on the positive solution phi to the expression:

phi\hat{ }2=phi+1

Using phi as a self-similarity constant, the present disclosure adopts a recognition scale defined as:

X_opt=phi/pi

The quantity X_opt is used herein as a theory-derived scaling anchor from which DNA-relevant length and energy relationships may be constructed. In the present disclosure, X_opt is not merely a symbolic constant; rather, it functions as the common origin point for the derived parameter set later used in the DNARP tuple and governing constructs. By deriving multiple DNA-relevant quantities from the same recognition scale, the disclosed framework provides a unified basis for relating sequence validity, helical geometry, coherence level, and predicted output within one computational system.

Recognition Science also provides a discrete recognition structure that, in some embodiments, may be described in terms of an eight-step or 8-tick periodic framework. In the present disclosure, that discrete structure is used only to the extent needed to support the DNA-adapted coherence formulation and associated rate relationships. Thus, the 8-tick structure is not introduced as an independent object of the claimed invention, but as part of the theoretical basis for using discrete allowed coherence levels, discrete operator-governed validation, and scale-linked rate behavior in the DNARP system. Where appropriate, that discrete structure may also support the way in which the baseline rate parameter is later related to a coherence-derived frequency and to discrete admissible states in the output formulation.

Recognition Science additionally employs a recognition operator, sometimes denoted as R_hat in the underlying theory, from which a DNA-adapted operator may be defined for the present disclosure. The present invention does not require claiming the abstract recognition operator in isolation. Instead, the operator concept is carried forward only insofar as it supports the later definition of a DNA recognition operator used to evaluate whether a requested coherence-related state is admissible for a given candidate DNA configuration. Accordingly, the Recognition Science basis disclosed here establishes that candidate DNA designs may be evaluated not only by classical sequence and geometry constraints, but also by a recognition-operator-based coherence constraint. This provides a theoretical bridge for the later coherence validation operations described with respect to the coherence validation diagram (700).

Applied to DNA, the Recognition Science basis used herein treats the double helix as a recognition-structured domain in which multiple forms of admissibility can be evaluated within a common framework. Complementary nucleotide pairing contributes to sequence-level stability.

Helical pitch and groove ratio contribute to shape-level admissibility. Discrete coherence-related states contribute to energy-level admissibility and to predicted output behavior. The disclosed DNARP framework uses Recognition Science only to the extent necessary to tie these otherwise separate considerations together through a common parameter origin and a common validation logic. Thus, the invention is not limited to conventional sequence analysis, nor to isolated geometric modeling, nor to purely empirical expression prediction. Instead, it uses Recognition Science-derived relationships to define a formal DNA program representation whose components may be checked against common governing constraints.

Stated differently, the role of Recognition Science in the present disclosure is to provide a basis for why specific DNA-relevant constants, ranges, and operator-based checks are used in the disclosed computational workflow. The derived parameters introduced later are not arbitrary fitted parameters selected independently for each task. Rather, they are linked to a common scaling origin and are then used to constrain the sequence component, the shape component, and the energy component of a DNARP program. In this way, Recognition Science supplies the underlying theoretical coherence of the disclosed design language, while the DNARP system supplies the practical computational implementation. The result is a formal and executable framework in which a candidate DNA design may be represented, checked for syntactic and physical admissibility, subjected to structural and coherence validation, and used to generate a predicted functional output.

The present specification relies on the cost-function foundation, the phi-based scaling relation, the recognition scale X_opt, the discrete recognition structure to the extent relevant to DNA coherence modeling, and the operator concept to the extent relevant to defining a DNA-adapted validation construct. Other domains, predictions, or broader theoretical consequences that may exist elsewhere in Recognition Science are not required for understanding or practicing the disclosed DNARP invention and are therefore omitted from the present discussion. This restrained use of Recognition Science keeps the present disclosure focused on the concrete DNA programming language, the governing DNA parameter set, and the corresponding computational evaluation framework.

The foregoing Recognition Science basis is implemented in the present disclosure through the DNARP program tuple (200) shown in (FIG. 2) and through the later governing computational constructs illustrated in (FIG. 6), (FIG. 7), and (FIG. 8). More particularly, the theory-derived scaling relations provide the basis for later defining DNA-relevant parameters used in the tuple representation, the structural stability formulation, the coherence validation formulation, and the output-prediction formulation. Those later sections set forth the operative quantities, computational tests, and decision logic by which a candidate DNA program is evaluated in practice.

The disclosed DNARP framework employs a defined set of Recognition Science-derived DNA parameters that function as the principal quantitative anchors for sequence evaluation, shape validation, coherence-state definition, and predicted-output computation. These parameters are not introduced as disconnected empirical constants selected independently for different tasks.

Rather, they are disclosed as a coordinated set of DNA-relevant quantities derived from the common recognition scale X_opt=phi/pi, and they provide a common mathematical basis for the shape component, energy component, and downstream governing constructs of the DNARP program tuple (200) shown in (FIG. 2). In this way, the disclosed parameter set allows a candidate DNA design to be represented and evaluated using a unified framework rather than a collection of unrelated fitting rules.

A first quantity in the disclosed parameter set is the recognition scale X_opt, defined as:

X_opt=phi/pi

As described above, X_opt functions as the common derivational basis for the DNA-relevant length and energy cascades used by the present invention. In the present disclosure, X_opt is not merely a background theoretical constant; rather, it serves as the originating scale from which the characteristic DNA distance, preferred helical pitch, coherence-energy quantum, base-pairing energy scale, and baseline output rate are derived. By using X_opt as a single starting point, the disclosed system ties together the geometric and energetic constraints later imposed on candidate DNARP programs.

A second quantity is the characteristic DNA recognition length X_DNA, defined as:

X_DNA=L_Planck*(X_opt)\hat{ }(−90)

In one disclosed embodiment, X_DNA is approximately 13.6 angstrom. This quantity represents a characteristic DNA-related recognition length scale and is used as a structural reference in multiple parts of the disclosed framework. For example, X_DNA provides the length basis for the normalization of the helix coordinate in the coherence formulation, and it also participates in the derivation of the preferred helical pitch and in the definition of implementation constants later used in the structural stability and coherence operator constructs. Accordingly, X_DNA is not merely a reported constant, but a working parameter used by the disclosed evaluation pipeline.

A third quantity is the preferred helical pitch P_0, defined as:

P_0=X_DNA*(phi\hat{ }2)

In one disclosed embodiment, P_0 is approximately 35.6 angstrom. The preferred helical pitch P_0 provides a theory-derived reference value for the pitch-related portion of the shape component (220) of the DNARP tuple (200). As shown in (FIG. 2), the tuple may include a helical pitch parameter (250), and as shown in (FIG. 5), the shape evaluation subsystem (500) may evaluate a candidate helical pitch (520) relative to a permitted pitch range (560). In some embodiments, admissibility of a candidate pitch is determined by whether the pitch falls within a prescribed range around P_0, such as P in [30, 40] angstrom. In other embodiments, P_0 may be used more directly as the reference value appearing in later calculations, including the phase term used in the output prediction formulation. Thus, P_0 functions both as a geometric target and as a computational reference parameter.

A fourth quantity is the preferred groove-ratio reference G_0, defined as:

G_0=phi

In one disclosed embodiment, G_0 is approximately 1.618. The quantity G_0 supplies a preferred reference for the groove ratio parameter (260) of the shape component (220). As shown in (FIG. 5), the disclosed shape evaluation subsystem (500) may evaluate a groove ratio (550) against a permitted groove-ratio range (570), such as G in [1.5, 1.7], which is centered on or otherwise associated with G_0. The use of G_0 allows the groove geometry of a candidate DNA design to be evaluated relative to a theory-derived reference rather than an arbitrary design guess. In some embodiments, the candidate groove ratio need only fall within an allowed admissibility band. In other embodiments, closeness to G_0 may be used as an additional compatibility factor, particularly for candidate designs requesting higher coherence levels or higher predicted outputs.

A fifth quantity is the coherence-energy quantum E_coh, defined as:

E_coh=E_Planck*(X_opt)\hat{ }(101)

In one disclosed embodiment, E_coh is approximately 0.09 eV. The quantity E_coh serves as the fundamental coherence-energy increment for the energy component (230) of the DNARP tuple (200), and is represented in connection with the coherence energy level (270) shown in (FIG. 2). More particularly, the disclosed framework defines allowable coherence energy levels in terms of integer multiples of E_coh, such that a candidate design may request or encode a coherence state E_n=n*E_coh for an allowed integer n. As further illustrated in the coherence validation diagram (700) of (FIG. 7), the discrete coherence energy level (750) may be validated against an allowed coherence state set (740). Thus, E_coh is the foundational energetic quantity that permits the energy component to be represented in discrete, computable form.

A sixth quantity is the base-pairing energy scale E_bp, defined as:

E_bp=E_coh*X_opt*2.5

In one disclosed embodiment, E_bp is approximately 11.1 kJ/mol. The parameter E_bp provides a DNA-relevant energy scale associated with base pairing and supports the stability logic used in the sequence component and structural admissibility analysis. Although the sequence stability score itself may be implemented in the disclosed system through a weighted contribution model, the present disclosure uses E_bp as part of the theory-derived basis for why complementary sequence interactions may be treated as quantifiable and why sequence-level thresholds are not arbitrary. In this sense, E_bp supports the logic by which CG-rich and AT-rich contributions may be distinguished in the sequence evaluation framework, while also reinforcing the relationship between the sequence component and the broader DNA recognition model.

A seventh quantity is the baseline output rate R_0, defined as:

R_0=(E_coh/h)*8*(phi\hat{ }(−60))

In one disclosed embodiment, R_0 is approximately 50 bases/second. The quantity R_0 serves as the baseline output-rate parameter for the energy component (230) and is represented in connection with the output rate parameter (280) shown in (FIG. 2). In the disclosed framework, R_0 provides the base rate from which candidate output rates are derived according to the selected coherence level. For example, a candidate having coherence index n may be associated with R=n*R_0, so that the output rate scales discretely with the coherence-energy level. This parameter is used later in the coherence and output-prediction constructs and is part of the final predicted functional output computation illustrated by the output prediction diagram (800) of (FIG. 8). Accordingly, R_0 is not merely a descriptive constant, but an operative parameter that directly participates in candidate-output computation.

Taken together, the foregoing quantities define the principal RS-derived DNA parameter set used by the present invention. The preferred helical pitch P_0 and preferred groove-ratio reference G_0 principally support the geometric and admissibility logic of the shape component (220), including the helical pitch parameter (250) and groove ratio parameter (260) represented in the tuple (200) and further evaluated in the shape evaluation subsystem (500) of (FIG. 5). The coherence-energy quantum E_coh and baseline output rate R_0 principally support the energy component (230), including the coherence energy level (270) and output rate parameter (280) represented in the tuple (200), the discrete coherence energy level (750) represented in the coherence validation diagram (700) of (FIG. 7), and the predicted functional output (820) represented in the output prediction diagram (800) of (FIG. 8). In this respect, the disclosed parameter set creates explicit continuity between the formal DNARP tuple representation and the later computational constructs used to evaluate candidate DNA programs.

In some embodiments, additional implementation constants may be used in connection with particular governing constructs. For example, constants such as kappa_DNA, lambda_DNA, and k_DNA may be introduced in later sections when describing the structural stability and coherence-validation formulations in operational detail. In the present section, however, those quantities need not be front-loaded as part of the general parameter set because their primary role is implementation-specific within later governing constructs rather than basic tuple-level representation. This keeps the present parameter discussion focused on the principal RS-derived DNA anchors that are most directly tied to the tuple-level language and to the major validation and output computations disclosed herein.

The disclosed RS-derived DNA parameter set therefore provides the quantitative backbone of the DNARP system. By deriving the principal DNA length, pitch, groove, coherence-energy, base-pairing-energy, and rate parameters from the common recognition scale X_opt, the present invention supplies a unified parameter origin for the representation and evaluation of candidate DNA designs. This allows sequence, shape, and energy to be treated as related parts of one formal and executable system rather than as separate categories addressed by disconnected tools. The resulting framework supports deterministic validation of candidate programs, structured comparison of alternative designs, and predicted functional output computation using a common set of disclosed, theory-derived DNA parameters.

The disclosed DNARP framework provides a formal language in which each candidate DNA design is represented as a three-component tuple, D=(S, H, E). In the present disclosure, this tuple is not merely a shorthand notation or a descriptive summary of a molecule. Rather, it is the foundational program structure by which a candidate DNA design is defined, stored, transmitted, parsed, evaluated, and, where applicable, iteratively modified within the disclosed computational system. A DNARP program therefore functions both as a formal design representation and as an executable or evaluable program representation for the DNARP Simulator. This dual role is significant because it allows a candidate DNA molecule to be handled as a structured computational object whose sequence-related, shape-related, and energy-related properties are jointly encoded in a single representation, rather than being distributed across disconnected tools or independently maintained parameter sets.

As shown in (FIG. 2), the DNARP program tuple (200) includes a sequence component (210), a shape component (220), and an energy component (230). These three components collectively define the candidate DNA program in a form suitable for deterministic computational evaluation.

The sequence component (210) captures the ordered sequence content of the candidate design, including the information needed to compute stability-related values and to test sequence-level admissibility. The shape component (220) captures helical-geometry information relevant to structural admissibility, including geometric quantities such as helical pitch and groove ratio. The energy component (230) captures the coherence-related energy state and associated rate information used in coherence validation and downstream output prediction. By combining these three components into the tuple (200), the disclosed language allows a candidate DNA design to be expressed as a complete design object whose principal admissibility and output-relevant characteristics are defined in a common format.

In this respect, the DNARP language differs from conventional representations that treat sequence, geometry, and functional prediction as separate domains. A conventional design workflow may specify a nucleotide string in one context, analyze helical geometry in another context, and estimate output or expression in yet another context using unrelated empirical tools. In contrast, the disclosed DNARP language encodes these categories together at the program level. The tuple D=(S, H, E) therefore serves as the formal interface between design definition and design evaluation. Once a candidate has been expressed in this form, the same representation may be used to perform syntax checking, threshold validation, structural stability evaluation, coherence validation, and predicted functional output computation. The language is therefore not limited to static description, but is expressly configured to support machine evaluation and decision-making.

The formal structure of the DNARP language also supports reproducibility and consistent implementation across different execution environments. Because the program is defined as an explicit tuple with defined component types, the system may receive, serialize, store, compare, or transform candidate DNA programs without changing the underlying design logic. For example, a DNARP program may be entered through a user interface, received through an application programming interface, retrieved from memory, or generated by an optimization routine, while still retaining the same tuple-level form. This is reflected in the high-level DNARP design and simulation system (100) shown in (FIG. 1), in which a DNARP program definition (120) is received and processed within the system architecture. Thus, the language disclosed herein is not tied to a single software layout or data-entry mode. Instead, it is defined at a level that permits implementation in local tools, remote services, automated design systems, or other computing arrangements capable of operating on the disclosed tuple structure.

The DNARP language is also executable in the sense that the tuple components are defined so as to feed directly into the staged computational workflow of the disclosed system. As shown in (FIG. 3), the DNARP evaluation method (300) includes a tuple parsing step (320) in which the candidate program is interpreted in accordance with the structure of D=(S, H, E). The parsed components are then used as inputs to subsequent evaluation operations. Thus, the language is intentionally designed so that its syntax corresponds to the operational needs of the simulator. The sequence component supplies the information used for sequence stability computation and threshold comparison. The shape component supplies the information used for geometric admissibility and structural stability analysis. The energy component supplies the information used for coherence-level validation and output-rate computation. Because the tuple is structured around the later stages of the evaluation method (300), the language itself is an enabling part of the computational framework rather than an abstract labeling convention.

In one implementation, the sequence component, the shape component, and the energy component may each be represented using structured fields, typed entries, or equivalent machine-readable forms. For instance, the sequence component may be represented as an ordered codon list, a nucleotide string, a grouped sequence block representation, or another sequence encoding sufficient to compute the disclosed sequence metrics. The shape component may be represented as a parameter pair, record, or other data structure containing at least helical pitch and groove-ratio information. The energy component may be represented as a discrete coherence state together with an associated output-rate value or derivable rate parameter. These forms may vary by implementation, but the logical structure remains the same: the DNARP program is treated as a unified candidate object having three defined component classes corresponding to S, H, and E. This implementation flexibility preserves the formal language while allowing different software embodiments to use arrays, objects, records, tables, or other internal data structures.

The tuple representation additionally supports comparison, validation, and optimization across multiple candidate designs. Because each candidate design is encoded in the same formal structure, different DNARP programs may be screened against common admissibility criteria, ranked by predicted output, or modified in an iterative design loop without changing the evaluation framework. A candidate having a different sequence but the same shape and energy targets may be represented as a different instance of D=(S, H, E). Likewise, a candidate having the same sequence but a different coherence target or different geometric parameters may be represented as another tuple instance. This provides a unified design language for both single-candidate evaluation and multi-candidate exploration. In some embodiments, the tuple form may also be used as the basic storage unit in a candidate library, an optimization workflow, or a cloud-based evaluation service.

The DNARP language is also intentionally structured to support objective validity checks. Each component of the tuple corresponds to a class of constraints or admissibility rules that may be evaluated independently and jointly. The sequence component may be tested against sequence-level stability criteria. The shape component may be tested against geometry-related admissibility ranges and structural stability conditions. The energy component may be tested against discrete coherence-state constraints and associated rate relationships. Because these checks are all grounded in the same tuple representation, the system can determine whether a candidate is invalid due to a deficiency in sequence content, geometric structure, coherence-level selection, or any combination thereof. This increases determinacy and improves the ability of the disclosed system to provide pass/fail decisions, identify failure modes, and support structured refinement of candidate designs.

In this way, the formal language disclosed herein is central to the invention. The DNARP program tuple (200) shown in (FIG. 2) is not merely a conceptual diagram, but a representation that organizes the technical inputs required for the disclosed DNA design and simulation framework. The DNARP program definition (120) shown in (FIG. 1) represents the manner in which such a tuple may be received or defined within the system (100), and the tuple parsing step (320) shown in (FIG. 3) represents the operational bridge by which the formal representation is transformed into executable evaluation steps within the DNARP evaluation method (300). Accordingly, the disclosed DNARP language provides a formal, machine-usable, and evaluation-ready representation of candidate DNA designs, enabling the later disclosed sequence analysis, shape validation, structural stability computation, coherence validation, and predicted functional output determination to proceed from a common and unified program definition.

The sequence component (210) provides the ordered sequence-content portion of a DNARP program tuple (200) and supplies the sequence-level information used by the disclosed system to determine whether a candidate DNA design satisfies the required stability and admissibility constraints for further evaluation. In the disclosed framework, the sequence component is not limited to a bare nucleotide listing. Rather, it is a structured sequence representation that may be expressed at one or more levels of granularity sufficient to support deterministic parsing, contribution counting, threshold comparison, and downstream compatibility analysis. As shown in (FIG. 2), the sequence component (210) may include a sequence representation (240) that encodes the ordered sequence content of the candidate DNA program in a form suitable for machine evaluation. In different embodiments, the sequence representation (240) may be provided as an ordered codon list, a nucleotide string, a grouped sequence-block representation, a functional-domain representation, or another structured arrangement of sequence units capable of supporting the disclosed evaluation operations.

In one implementation, the sequence component (210) is represented as an ordered list of codons. This codon-level representation may be useful where the candidate DNA design is directed to a coding region, an expression-related construct, or another embodiment in which triplet organization is relevant to design selection or iterative optimization. In another implementation, the sequence component (210) is represented directly as a nucleotide-level string, which may be advantageous for regulatory elements, non-coding regions, hybrid constructs, logic-gate segments, or other embodiments where codon framing is not the principal organizing feature. In still other embodiments, the sequence component (210) may be organized into grouped sequence blocks corresponding to functional units, sub-domains, motifs, regulatory segments, spacer regions, trigger regions, or other defined portions of the candidate design.

These alternative representations are all consistent with the disclosed framework so long as the sequence component (210) preserves the ordered sequence information needed to compute the disclosed sequence metrics and to test the candidate against the required sequence-level constraints.

The sequence component (210) is used by the system to evaluate sequence-level stability through a weighted contribution model in which complementary base-pair content is translated into a sequence stability score. In one disclosed embodiment, the sequence evaluation subsystem (400) shown in (FIG. 4) receives a codon sequence input (410) or an equivalent sequence input, determines a CG pair count (420) and an AT pair count (430), and applies those quantities to a stability scoring engine (440) to compute a sequence stability score (450). An exemplary ASCII-safe expression for this computation is:

S_stab=1.5*(number of CG pairs)+1.0*(number of AT pairs)

This expression reflects the disclosed weighting in which CG pairs contribute more strongly to sequence stability than AT pairs. The distinction is useful because the disclosed framework does not treat all complementary sequence contributions as identical for purposes of candidate validation. Rather, the sequence component (210) encodes a candidate's ordered sequence content in a manner that allows the system to distinguish stronger and weaker complementary contributions and to convert those contributions into a single computable stability quantity. The resulting sequence stability score (450) may then be compared against one or more admissibility thresholds to determine whether the candidate sequence is sufficient for continued evaluation.

In one expressly disclosed embodiment, the sequence component (210) must satisfy a minimum threshold on a per-functional-unit basis. An exemplary threshold condition is:

S_stab>=3 per functional unit

This threshold may be evaluated by the threshold comparator (470) of the sequence evaluation subsystem (400) against a sequence validity threshold (460). If the sequence stability score (450) fails to satisfy the required threshold, the candidate DNA program may be rejected at the sequence level before more computationally intensive downstream analyses are performed. In this way, the sequence component (210) acts as an early-stage admissibility gate within the overall DNARP evaluation framework. The sequence-level thresholding is useful not only for filtering out weak or under-supported candidates, but also for ensuring that later shape-level, structural, and coherence-level operations are applied only to candidate designs having sufficient sequence-level support under the disclosed framework.

In some embodiments, the disclosed system may additionally recognize a non-negative stability condition, such as:

S_stab>=0

This non-negative condition may be used as a minimal syntactic or computational sanity condition, while the higher threshold condition, such as S_stab>=3 per functional unit, is used as the substantive admissibility criterion for progression through the method. Thus, different thresholds may serve different purposes within the same implementation. A first threshold may ensure that the sequence component (210) is parseable and computationally valid, while a second threshold may determine whether the candidate is sufficiently stable to qualify as a valid DNARP program for later structural and coherence-based evaluation.

The term functional unit, as used in connection with the sequence component (210), may refer to a codon set, a nucleotide segment, a grouped sequence block, a regulatory sub-region, or another defined sequence portion selected as the unit of analysis for threshold application. The disclosed framework does not require that every candidate be treated as a single undivided sequence for all purposes. Rather, the sequence representation (240) may be segmented into one or more functional units, each of which may be scored separately, screened separately, or compared separately depending on implementation needs. For example, a candidate coding construct may be segmented into codon-grouped regions, a regulatory construct may be segmented into motif-containing sub-regions, and a DNA computing construct may be segmented into trigger, gate, and supporting regions. The sequence component (210) therefore supports both whole-sequence evaluation and sub-program evaluation within a common formal structure.

This segmentation capability is useful because different candidate DNA programs may contain sequence portions that play different technical roles. A therapeutic construct may include one region directed to coding content and another region directed to regulatory control. A promoter-oriented candidate may include multiple variant subregions targeted for tuning. A DNA logic construct may include a trigger-responsive region and a gate-forming region. By permitting the sequence representation (240) to be organized into functional blocks or grouped sequence units, the disclosed DNARP language allows the same candidate DNA program to be evaluated at the level most appropriate for the design problem at issue. The sequence component (210) is therefore not restricted to a single input style, but is adaptable to different sequence organizations while preserving the same underlying scoring and threshold logic.

In one embodiment, the disclosed system may receive sequence information through a direct codon-entry interface, a nucleotide-entry interface, an uploaded design file, an application programming interface, a stored candidate record, or an automated design-generation routine.

Regardless of the entry mode, the sequence evaluation subsystem (400) may normalize the incoming sequence representation into a machine-usable internal form suitable for counting operations and threshold testing. Such normalization may include codon parsing, case normalization, validation of nucleotide characters, segmentation into functional blocks, mapping of grouped sequence units to internal records, or conversion of alternative sequence-input formats into a common representation. This normalization step allows the sequence component (210) to remain logically consistent across different software implementations and different modes of candidate generation.

The disclosed framework also permits optional codon substitution or sequence modification operations to be applied to the sequence component (210). For example, once the sequence stability score (450) is computed, the system may alter one or more codons or nucleotides to increase CG content, redistribute complementary contributions, improve local sequence stability, or otherwise move the candidate toward threshold satisfaction or toward a target output objective. Such modification operations may occur in an iterative design loop and may be guided by the same sequence-level rules used for initial validation. The sequence component (210) therefore supports not only passive evaluation of a fixed candidate, but also active refinement of a candidate sequence under disclosed scoring criteria. In this way, the sequence representation (240) may function as both an input object and an optimization target within the larger DNARP system.

In addition to raw pair counting, the sequence component (210) may in some embodiments be evaluated for compatibility with sequence length, periodicity, or structural fit requirements associated with the candidate's intended geometry or coherence level. For example, sequence length or local block length may be tested for compatibility with a desired helical pitch, repeating structural interval, or another disclosed admissibility condition. Such checks need not replace the primary stability score, but may supplement it as additional sequence-level filters. Thus, the sequence component (210) may encode not only compositional information, but also organizational information relevant to whether the candidate sequence can coherently participate in the broader sequence-shape-energy framework of the DNARP tuple (200).

In some embodiments, stricter sequence-level requirements may be applied to candidates associated with higher requested coherence states or higher target outputs. For example, a candidate sequence that nominally passes a baseline threshold may still be deemed insufficient for a higher-level coherence request if the sequence stability score (450) is too close to the threshold or if the sequence content fails a more demanding compatibility criterion. This is consistent with the disclosed framework in which the sequence component (210), shape component, and energy component are not wholly independent, but may be jointly assessed for compatibility. Thus, the sequence component (210) may contribute not only a standalone pass/fail determination, but also a compatibility input to later stages of structural stability evaluation, coherence validation, and output prediction.

The sequence evaluation subsystem (400) shown in (FIG. 4) is therefore one exemplary implementation of how the sequence component (210) is used in practice. The codon sequence input (410) provides the incoming sequence data, the CG pair count (420) and AT pair count (430) provide the compositional measurements, the stability scoring engine (440) computes the sequence stability score (450), and the threshold comparator (470) compares that score against the sequence validity threshold (460). Although (FIG. 4) illustrates one specific arrangement of sequence-evaluation elements, the disclosed invention is not limited to that exact software arrangement. Equivalent counting engines, scoring modules, sequence parsers, or threshold-testing routines may be used so long as they implement the same technical logic of deriving a stability quantity from the sequence component and testing that quantity against one or more admissibility criteria.

The sequence component (210) is therefore a foundational part of the DNARP program tuple (200). It provides the ordered sequence data from which the system determines whether a candidate DNA design possesses sufficient sequence-level support to enter and proceed through the disclosed evaluation framework. By permitting codon-level, nucleotide-level, and grouped-block representations, by providing an explicit weighted stability formulation, by supporting threshold-based admissibility testing, and by enabling segmentation and iterative refinement, the sequence component (210) allows sequence information to be treated as a formal, machine-usable, and evaluation-ready component of a candidate DNA program rather than as an unstructured biological string. In this way, the disclosed sequence component (210) forms the sequence-level basis for the later shape validation, structural stability computation, coherence validation, and predicted functional output operations of the DNARP system.

The shape component (220) provides the geometric portion of a DNARP program tuple (200) and defines the helical-form constraints under which a candidate DNA design is treated as structurally admissible within the disclosed framework. In the present disclosure, the shape component is represented as H=(P, G), where P is helical pitch and G is groove ratio. This representation allows the disclosed DNARP language to encode not only sequence content and coherence state, but also the principal helix-geometry parameters needed to evaluate whether a candidate DNA design is compatible with the structural assumptions of the simulator. As shown in (FIG. 2), the shape component (220) may include a helical pitch parameter (250) and a groove ratio parameter (260), thereby allowing a candidate DNARP program to carry explicit geometry-related values as part of the formal tuple representation.

In operational terms, helical pitch P may be understood as the axial distance associated with one helical turn or other corresponding repeating helical interval used by the disclosed system to characterize the candidate DNA helix. In the present framework, pitch is not included as a purely descriptive biological quantity. Rather, it is a computational parameter used to determine whether the candidate DNA design is geometrically admissible, structurally stable, and compatible with later coherence and output calculations. The disclosed system therefore treats P as a design variable or input parameter that may be specified directly, estimated from a candidate structure, selected from a stored candidate record, generated by a design routine, or otherwise associated with the candidate DNA program before or during evaluation. As shown in (FIG. 5), a helical pitch (520) may be evaluated within a shape evaluation subsystem (500) in relation to a DNA helix (510) and to a permitted pitch range (560).

The groove ratio G may be understood as a parameter characterizing the relative geometry of the major groove (530) and minor groove (540) of the DNA helix (510). In one implementation, G is treated as a dimensionless ratio representing the relative proportions of the major and minor groove structure. In the present disclosure, groove ratio is not merely included for descriptive completeness; it functions as an admissibility parameter used to determine whether the candidate DNA design conforms to the disclosed geometric framework. As shown in (FIG. 5), the groove ratio (550) may be evaluated against a permitted groove-ratio range (570) by the shape evaluation subsystem (500). By explicitly incorporating groove ratio into the shape component (220), the disclosed DNARP language allows the candidate DNA program to encode a second geometry-related constraint in addition to helical pitch, thereby avoiding overreliance on sequence information alone.

In one disclosed embodiment, the shape component satisfies the expression:

H=(P,G)

with the following exemplary constraints:
P in [30, 40] angstrom
G in [1.5, 1.7]

These ranges provide objective admissibility bounds for the shape component and are used by the disclosed system as syntactic and computational constraints during DNARP program evaluation. A candidate DNA program whose helical pitch falls outside the disclosed pitch interval may be rejected as geometrically non-admissible, and a candidate whose groove ratio falls outside the disclosed groove-ratio interval may likewise be rejected or flagged as incompatible with the disclosed structural framework. In this way, the shape component (220) functions as a geometry-based gate within the broader DNARP evaluation process, analogous to the way the sequence component provides a sequence-based gate.

In some embodiments, the admissibility ranges for P and G may be interpreted relative to preferred reference values derived from the Recognition Science-based parameter set previously described. More particularly, the disclosed framework may treat the pitch range as centered on the preferred helical pitch P_0, and may treat the groove-ratio range as centered on the preferred groove-ratio reference G_0. In one disclosed embodiment, P_0 is approximately 35.6 angstrom, and G_0 is approximately 1.618. Accordingly, the shape component (220) may be evaluated not only on the basis of whether the candidate values fall within an allowed interval, but also on the basis of how closely those values conform to the preferred geometric references derived from the common Recognition Science scaling framework. Such closeness criteria may be used, for example, to distinguish minimally admissible candidates from more strongly conforming candidates, or to impose more selective admissibility conditions where higher coherence states or higher target outputs are sought.

The disclosed shape formulation therefore supports multiple levels of geometric evaluation. At a first level, the shape component (220) may be tested against fixed admissibility intervals such as P in [30, 40] angstrom and G in [1.5, 1.7]. At a second level, the candidate pitch and groove ratio may be compared to preferred values P_0 and G_0 to determine geometric closeness, conformity score, or compatibility rank. At a third level, these geometric values may be used as inputs to later governing constructs, including structural stability evaluation and coherence validation. The shape component is therefore not limited to a static range-checking role. Instead, it provides geometry-related information that can be used across the full DNARP pipeline, from early admissibility filtering through later physics-based and output-related evaluation stages.

The shape evaluation subsystem (500) shown in (FIG. 5) is one exemplary implementation of how the shape component (220) may be processed. In such an embodiment, the DNA helix (510) provides a geometric model or representation of the candidate helix. The helical pitch (520), major groove (530), and minor groove (540) may be identified, measured, estimated, assigned, or otherwise associated with the candidate design. The groove ratio (550) may then be determined from the relative groove geometry, and the resulting pitch and groove-ratio values may be compared against the permitted pitch range (560) and permitted groove-ratio range (570).

Although (FIG. 5) illustrates one representative geometry-evaluation arrangement, the invention is not limited to any specific rendering or internal software layout. Equivalent parameter-estimation routines, geometric parsers, helix-model representations, or comparator modules may be used so long as they implement the disclosed technical logic of encoding and evaluating P and G as part of the DNARP tuple.

The shape component (220) may be populated in different ways depending on the embodiment.

In some implementations, pitch and groove-ratio values are directly entered by a user or supplied through an application programming interface as part of a candidate DNARP program definition.

In some implementations, these values are inferred from a candidate structural model, a stored structural template, a predicted helical representation, or another geometry derivation process. In still other implementations, the disclosed system may generate one or both values during iterative design, such as when exploring alternative candidate constructs that vary not only by sequence content but also by target helical geometry. The formal structure of the shape component therefore accommodates direct specification, inferred estimation, templated reuse, and generated geometry assignments, while preserving the same tuple-level representation.

The shape component (220) may also interact with sequence-level and energy-level constraints rather than operating in isolation. For example, a candidate may satisfy the sequence stability threshold yet still be rejected if its pitch or groove ratio falls outside the permitted ranges. Conversely, a candidate with a geometrically admissible shape may still fail overall evaluation if its sequence stability is insufficient or if its requested coherence state is not valid. In some embodiments, the disclosed system may apply tighter geometric requirements to candidates requesting higher coherence levels. Thus, a candidate associated with a higher n value may be required not merely to fall within the broad range for P and G, but to lie closer to P_0 and G_0 than would be required for a lower-level candidate. This is consistent with the disclosed framework in which sequence, shape, and energy are coordinated components of a single design language rather than wholly independent variables.

The helical pitch parameter is also used downstream in the disclosed structural stability formulation. In the disclosed framework, the shape component supplies the pitch term used in the DNA Lagrangian and in the resulting stability condition. More particularly, the structural stability condition later described for C(r) depends on the helical pitch P, and the disclosed stable-solution form is satisfied when P remains within the disclosed admissible range. Thus, the pitch information carried by the shape component (220) is not merely screened and discarded after an early syntax check. Instead, it becomes an operative parameter in the later structural evaluation steps of the DNARP system. Similarly, the disclosed coherence and output-related constructs may also depend on the candidate geometry, including pitch-related phase terms and geometry-linked admissibility conditions. The shape component therefore forms a bridge between initial tuple definition and later governing-construct computations.

The groove ratio parameter likewise supports more than a simple geometric description. Because the groove ratio (550) represents a structural aspect of the DNA helix (510) that is tied to the overall admissibility of the candidate design, it may be used as a compatibility factor when comparing candidate molecules that otherwise share similar sequence properties. For instance, two candidate DNARP programs may have similar sequence stability values yet differ in groove-ratio conformity, and the disclosed system may treat the candidate closer to G_0 as more compatible with a selected coherence level or target output. In this manner, the groove ratio parameter (260) allows the disclosed framework to distinguish between candidates that might appear similar under sequence-only evaluation but differ in geometry-based quality or admissibility.

In some embodiments, the shape component may additionally support tolerance-band implementations rather than only strict interval limits. Thus, the pitch or groove-ratio values may be represented, stored, or processed with explicit tolerances, uncertainty bands, or discretized bins, especially where the values are generated numerically or inferred from another model. A candidate may be treated as admissible if its evaluated P and G values fall within specified tolerance windows around the disclosed target ranges or preferred reference values. Such tolerance-based processing remains fully consistent with the disclosed formal language because the shape component is defined by the presence of the geometry parameters and their evaluation against disclosed admissibility criteria, rather than by any one required numerical routine.

The shape component (220) is therefore a foundational part of the DNARP program tuple (200).

It gives the disclosed language a formal mechanism for encoding helix geometry in the candidate DNA program and allows geometric admissibility to be treated as an explicit design constraint rather than an informal downstream consideration. By representing a candidate shape as H=(P, G), by defining objective admissibility ranges for helical pitch and groove ratio, by permitting comparison to preferred values P_0 and G_0, and by enabling downstream use of the pitch and groove-related parameters in structural and coherence-related calculations, the disclosed shape component (220) allows DNA geometry to function as a formal, machine-usable, and evaluation-ready element of the DNARP framework. In this way, the shape component (220), together with the sequence component and the energy component, enables a candidate DNA design to be represented and evaluated as a unified computational program rather than as a disconnected set of biological descriptors.

The energy component (230) provides the coherence-state and rate-related portion of a DNARP program tuple (200) and defines the discrete energetic state under which a candidate DNA design is evaluated for coherence admissibility and predicted functional output. In the disclosed framework, the energy component is represented as E=(E_n, R), where E_n is a coherence-related energy level and R is an associated output or expression rate. This representation allows the disclosed DNARP language to encode, in a machine-usable form, not only the existence of an energetic state, but also the rate quantity that is used in downstream output computation. As shown in (FIG. 2), the energy component (230) may include a coherence energy level (270) and a predicted output rate (280), thereby allowing the candidate DNARP program to carry explicit energy-state and rate values as part of the formal tuple representation.

In the present disclosure, the coherence-related energy level E_n is treated as a discrete quantity rather than a continuously arbitrary value. More particularly, the disclosed system defines allowable coherence states in terms of integer multiples of the coherence-energy quantum E_coh, such that:

E_n=n*E_coh

where n is an allowed integer coherence index. In the expressly described embodiment, the allowed index values are:
n in {1, 2, 3}

Thus, the energy component (230) is not satisfied merely by any asserted energy assignment.

Instead, the candidate DNA program is expected to specify or imply a discrete coherence level that is consistent with the allowed state structure of the disclosed framework. This discrete-state formulation is important because it allows coherence validation to be carried out objectively, using state-based admissibility logic rather than an unconstrained energetic estimate. As further supported by the coherence validation diagram (700) of (FIG. 7), the discrete coherence energy level (750) may be evaluated against an allowed coherence state set (740) to determine whether the requested state is valid for the candidate design.

In one disclosed embodiment, the baseline coherence-energy quantum is given by E_coh, previously introduced as an RS-derived DNA parameter. Accordingly, the first coherence level may be represented by E_1=1*E_coh, the second coherence level by E_2=2*E_coh, and the third coherence level by E_3=3*E_coh. In an implementation where E_coh is approximately 0.09 eV, these levels may correspond approximately to 0.09 eV, 0.18 eV, and 0.27 eV, respectively. The disclosed invention is not limited to these numerical examples as exact universal requirements, but these values provide one concrete embodiment of how the energy component (230) may be instantiated for a candidate DNARP program. By defining E_n in this manner, the disclosed system makes the requested coherence level explicit, reproducible, and directly testable during later validation steps.

The second quantity in the energy component (230) is the associated rate R. In the disclosed framework, R represents a predicted output rate, expression-related rate, or other functionally relevant rate quantity associated with the selected coherence level. The rate is likewise treated as a structured program value rather than an unconstrained label. In one disclosed embodiment, the rate satisfies:

R=n*R_0

where R_0 is the baseline output-rate parameter derived from the common RS-based parameter set. In an implementation where R_0 is approximately 50 bases/second, the allowed rates associated with the expressly described three-level embodiment may be approximately 50 bases/second, 100 bases/second, and 150 bases/second for n=1, n=2, and n=3, respectively.

This discrete rate scaling is useful because it ties the rate quantity directly to the selected coherence state and prevents the energy component (230) from becoming a free-floating or independently chosen output label. Instead, the disclosed framework uses the coherence index to govern both the energy level and the associated rate in a coordinated manner.

In the expressly described three-level embodiment, the energy component may therefore satisfy both of the following relations:

E_n=n*E_coh

R=n*R_0

with:
n in {1, 2, 3}
   and, in one example implementation:
R<=150 bases/second

The upper bound on R follows from the highest expressly described state n=3 and provides an objective ceiling for the three-level embodiment. This upper limit is useful for determinacy because it defines the maximum rate associated with the currently disclosed discrete-state implementation. In some embodiments, the system may use this upper bound as part of a validity check to ensure that a candidate DNA program does not request a rate inconsistent with the permitted coherence-state structure. Thus, a candidate requesting R above the maximum rate associated with the allowed discrete state set may be rejected, flagged for correction, or treated as incompatible with the currently selected embodiment of the disclosed system.

The energy component (230) is not merely a post-processing output container. Rather, it is part of the candidate DNARP program itself and therefore participates in the evaluation logic from an early stage. When a candidate tuple is parsed, the disclosed system may determine whether the specified or selected E_n corresponds to one of the allowed coherence states and whether the associated rate R is consistent with the selected n. For example, if a candidate requests the second coherence state, the system may verify that the candidate's coherence energy level equals 2*E_coh and that its associated rate equals 2*R_0. If these conditions are not satisfied, the candidate may be rejected before downstream calculations are completed. In this way, the energy component (230) acts as an admissibility-bearing part of the formal language rather than as a result appended after all other checks are complete.

The coherence index n provides a convenient way to describe increasingly energetic or increasingly output-capable candidate states within the disclosed framework. In one embodiment, n=1 represents a baseline state. This baseline state may correspond to a minimally valid or minimally enhanced coherence condition, and it may be associated with the lowest allowed coherence energy level and the baseline rate R_0. In one embodiment, n=2 represents an enhanced state relative to the baseline, with both higher coherence energy and higher associated rate. In one embodiment, n=3 represents the highest expressly described state and may be associated with the maximum expressly described rate within the current disclosure.

These discrete levels provide an orderly and deterministic way to encode different candidate operating states without requiring the designer or the system to search over a continuous unconstrained energy axis.

Although the disclosed framework expressly describes the three-level implementation n in {1, 2, 3}, the disclosed three-level embodiment provides a concrete implementation in which the coherence state, associated energy level, and associated rate are defined in a deterministic and machine-usable manner. It supplies definite state values, definite scaling relationships, and a definite upper rate bound. This keeps the disclosed energy component (230) grounded in an operational structure that can be evaluated and implemented without introducing unnecessary breadth untethered from the disclosed examples and governing constructs.

The relationship between the energy component (230) and the rest of the tuple is also significant. The disclosed framework treats sequence, shape, and energy as coordinated components rather than independent variables. Thus, the selected coherence state may be subject to compatibility constraints involving the sequence component and the shape component. A candidate may satisfy the formal energy equations for a requested n, yet still be rejected if its sequence stability is insufficient or if its geometry is not sufficiently compatible with the requested state. For example, higher coherence states may require stronger sequence-level support or closer conformance to preferred geometric values. In one embodiment, n=2 is permitted when S_stab>3, and n=3 is permitted when S_stab>=4.5, with higher n optionally requiring the shape component to lie closer to preferred values P_0 and G_0 (e.g., within narrower tolerance bands). In this manner, the energy component (230) is part of a coordinated admissibility framework in which higher coherence capability may be conditioned on stronger support from the sequence and shape components.

The energy component (230) also provides the direct input quantities used in later coherence validation operations. As shown in (FIG. 7), allowed coherence states may be validated using the disclosed coherence-validation framework, and as shown in (FIG. 8), validated candidate states may be used for downstream predicted output computation. More particularly, the allowed coherence state set (740) and discrete coherence energy level (750) in the coherence validation diagram (700) provide the figure-level structure by which a requested E_n may be tested for state validity. Once the requested state is validated, the same selected coherence level and associated rate may be carried forward into the output-prediction formulation that generates the predicted functional output (820) in the output prediction embodiment (800). Thus, the energy component (230) forms the direct bridge between tuple-level state selection and the later operator-based and transform-based computational stages of the disclosed system.

In one implementation, the energy component may be provided as a pair of explicit values, such as a stored coherence level and a stored rate. In another implementation, the energy component may be provided by specifying only the coherence index n, after which the system derives E_n and R internally using the disclosed equations. In another implementation, the system may receive a requested energy level and infer the corresponding index if the value matches an allowed discrete state within a tolerance band. In still other implementations, the system may receive a target output goal and determine which coherence level should be tested first. These alternatives remain consistent with the disclosed framework because the defining feature of the energy component is not the user-interface format, but the fact that the candidate program ultimately carries or implies a discrete coherence state and an associated rate governed by the disclosed relationships.

The energy component (230) may also be stored, transmitted, or compared in candidate libraries, optimization routines, or automated design workflows. Because the candidate DNA design is represented as a tuple, different candidates may be compared not only by sequence content and geometry, but also by the coherence-state and rate selections encoded in the energy component.

A first candidate may be designed for baseline operation at n=1, whereas a second candidate may be designed for enhanced output at n=2, and the disclosed system may compare these candidates under the same sequence and shape evaluation framework. Likewise, an iterative design routine may hold the sequence and shape components fixed while exploring whether a higher coherence level is admissible, or may hold the energy target fixed while modifying sequence and geometry to support that target. This flexibility further illustrates that the energy component (230) is not an afterthought; it is a formal part of the candidate program definition that supports structured design exploration.

The energy component (230) therefore provides the discrete coherence-state and associated rate information required for the disclosed DNARP framework to operate as a unified formal language and evaluation system. By representing energy as E=(E_n, R), by defining E_n as an integer multiple of E_coh, by defining R as the corresponding multiple of R_0, by expressly disclosing the three-level embodiment n in {1, 2, 3}, and by tying higher-energy requests to sequence and shape compatibility, the disclosed framework allows the candidate DNA program to encode a coherence-governed operating state in a form that is machine-usable, objectively testable, and directly connected to later coherence validation and predicted-output computation. In this way, the energy component (230), together with the sequence component and the shape component, completes the formal tuple representation by which a candidate DNA design is represented and evaluated under the DNARP system.

The sequence component (210), the shape component (220), and the energy component (230) of the DNARP program tuple (200) are coordinated components of a single formal design representation and are evaluated together to determine whether a candidate DNA program is admissible within the disclosed framework. Although each component carries its own parameters and constraints, the disclosed DNARP system does not treat those components as wholly independent variables that may be selected in isolation without regard to the rest of the tuple. Instead, the system evaluates whether the sequence, geometry, and coherence-state selections are mutually compatible in light of the disclosed thresholds, permitted ranges, and state constraints. This coordinated treatment is important because the disclosed invention is directed to a unified DNA programming language and simulation framework, rather than to separate tools for sequence scoring, geometric screening, and rate estimation.

In one aspect, a candidate DNA program is invalid if the sequence component fails its required sequence-level admissibility threshold, even if the shape component and energy component are otherwise nominally within allowed limits. Thus, where the disclosed system computes a sequence stability score and determines that the stability score is below the required threshold for the relevant functional unit, the candidate may be rejected without regard to whether the helical pitch, groove ratio, or requested coherence state would otherwise satisfy their respective constraints. This provides an objective rule by which insufficient sequence support is treated as a threshold failure that blocks downstream acceptance of the candidate tuple (200). As reflected in the DNARP evaluation method (300), such a failure may occur at the sequence stability computation step (330) and may lead to candidate rejection at the candidate output step (380).

In another aspect, a candidate DNA program is invalid if the shape component (220) falls outside the permitted geometric constraints, even where the sequence component and energy component appear acceptable. Thus, a candidate having a valid sequence stability score may still be rejected if its helical pitch is outside the disclosed permitted range or if its groove ratio is outside the disclosed permitted range. In the present framework, geometric admissibility is not optional or secondary to sequence validity. Rather, the shape component forms an independent gate that must be satisfied for the overall tuple to remain eligible for further evaluation. This means that a candidate may fail because its geometric parameters are incompatible with the disclosed framework, even though its sequence content and requested coherence state are otherwise plausible. As reflected in the DNARP evaluation method (300), this determination may be made at the shape constraint validation step (340), after which an out-of-range candidate may be halted and reported as invalid at the candidate output step (380).

In another aspect, a candidate DNA program is invalid if the requested coherence level encoded in the energy component (230) is not consistent with the disclosed discrete-state structure or is not compatible with the candidate's validated configuration. Thus, the disclosed framework does not permit a candidate to request an arbitrary coherence energy level or arbitrary associated output rate. Instead, the requested energy state must conform to the allowed discrete coherence-state logic of the system, and the candidate may be rejected if the requested state falls outside the allowed state set or if the corresponding rate does not match the requested level. Moreover, even where the formal discrete-state relationship is satisfied, the requested coherence level may still be rejected if the candidate's sequence and geometry do not provide sufficient support for that level. As reflected in the DNARP evaluation method (300), such determinations may be made at the coherence validation step (360), with invalid candidates again being rejected at the candidate output step (380).

The disclosed framework therefore imposes compatibility rules that extend beyond isolated threshold checks. In one embodiment, a higher requested coherence state may require a higher sequence stability score than would be required for a lower requested coherence state. In another embodiment, a higher requested coherence state may require the shape component to conform more closely to the preferred geometric references used by the system than would be required for a lower requested coherence state. Thus, a candidate that is acceptable at a baseline coherence level may become unacceptable at a higher coherence level if the sequence component is not sufficiently strong or if the shape component is not sufficiently conforming. This compatibility logic is useful because it prevents the energy component from being chosen in a purely aspirational manner disconnected from the physical and structural support provided by the rest of the tuple. Instead, the requested coherence level is treated as an admissibility-bearing selection that must be justified by the candidate's sequence and geometry.

In this manner, the compatibility rules among the sequence component (210), the shape component (220), and the energy component (230) provide objective bounds for determining overall candidate validity. The tuple (200) shown in (FIG. 2) illustrates that these components exist within a common program representation, and the DNARP evaluation method (300) shown in (FIG. 3) illustrates that the candidate is evaluated through an ordered decision path in which sequence-related, shape-related, and coherence-related checks collectively determine whether the candidate proceeds or is rejected. Accordingly, the disclosed compatibility rules make clear that overall DNARP validity is based on coordinated admissibility across the tuple, not on isolated satisfaction of any one component alone. This provides definitional clarity, supports deterministic pass/fail logic, and helps ensure that a candidate DNA program accepted by the system represents a mutually compatible combination of sequence, shape, and energy selections rather than a merely partial or internally inconsistent design.

Structural stability evaluation is performed in the disclosed DNARP framework to determine whether a candidate DNA design maintains an admissible helix-associated base-pairing profile under the geometric conditions encoded by the candidate tuple. In the present disclosure, structural stability is not treated as a purely descriptive property inferred informally from sequence or shape alone. Rather, it is evaluated through a governing computational construct that receives candidate geometry information, applies a defined DNA Lagrangian, and determines whether the resulting stability behavior satisfies an objective tolerance-based acceptance condition. This structural stability evaluation corresponds to the structural stability evaluation step (350) in the DNARP evaluation method (300) of (FIG. 3), and is further illustrated in the structural stability evaluation diagram (600) of (FIG. 6), which includes the DNA Lagrangian (610), coordinate variable (630), stability solver (640), stability curve (650), and stability tolerance band (660).

In one disclosed embodiment, the governing construct used for structural stability evaluation is a DNA Lagrangian defined as:

L_DNA=(kappa_DNA/2)*(dC/dr)\hat{ }2-lambda_DNA*((1-C\hat{ }2)/2-0.1*C*cos(2*pi*r/P))

In this expression, C(r) denotes a base-pairing profile or base-pairing probability field along a helix coordinate r, P denotes the helical pitch obtained from the shape component (220), kappa_DNA is a rigidity-related parameter, and lambda_DNA is an energy-density-related parameter. The disclosed Lagrangian provides a formal way to evaluate whether a candidate DNA design remains structurally well-behaved under the geometric configuration specified by the candidate DNARP program. The Lagrangian therefore acts as the first governing construct by which the system moves beyond threshold checking of tuple syntax and into evaluation of dynamic or spatial structural admissibility.

The quantity C(r) may be understood as a spatially varying measure of base-pairing support, pairing coherence, or local structural integrity along the helix coordinate r. In one implementation, C(r) is treated as a normalized field whose idealized stable value is near unity across the domain of interest. Under this interpretation, deviations of C(r) away from unity correspond to instability, distortion, or reduced structural support within the candidate DNA design. The coordinate r may be treated as a position variable along the helix, for example measured in angstrom units or another selected length unit used consistently within the implementation. The helical pitch P enters directly into the cosine term and therefore links the structural stability evaluation to the shape component (220) of the DNARP tuple (200). This means that the geometry selected in the tuple is not merely screened at an earlier stage and discarded; instead, that geometry becomes an active input to the stability analysis itself.

In one disclosed embodiment, kappa_DNA is a rigidity-related constant representing the contribution of structural resistance to variation in the base-pairing profile. In one example implementation, kappa_DNA is approximately 1.56×10\hat{ }(−18) J*angstrom. Likewise, lambda_DNA is an energy-density-related constant associated with pairing support and the stabilizing influence of the helix-coupled potential term. In one example implementation, lambda_DNA is approximately 7.3×10\hat{ }7 J/m\hat{ }3. These values are provided as exemplary implementation constants and may be used directly, approximately, or within bounded tolerance intervals depending on the embodiment. The present disclosure does not require that every implementation use exactly one numerical solver, one coordinate discretization method, or one exact numerical precision, so long as the disclosed system applies the structural stability formulation in a manner that preserves the operative relationship among C(r), r, P, kappa_DNA, and lambda_DNA.

The disclosed DNARP system may evaluate structural stability by solving, numerically approximating, discretizing, or otherwise processing an Euler-Lagrange equation derived from L_DNA. In one implementation, the stability solver (640) shown in (FIG. 6) receives the DNA Lagrangian (610), the coordinate variable (630), and the candidate helical pitch supplied from the shape component, and then computes or approximates a resulting stability curve (650) corresponding to the behavior of C(r) over a selected domain. The selected domain may span all or part of the candidate DNA design, a representative interval corresponding to one or more helical turns, or another region chosen for structural evaluation. The solver may employ analytical approximation, numerical integration, finite-difference methods, finite-element methods, iterative relaxation, spectral approximation, or equivalent computational techniques suitable for obtaining a usable profile for C(r).

In one implementation, the coordinate variable r is discretized as a grid r_i with step size Δr selected to resolve helix-coupled periodicity associated with pitch P (e.g., a step size chosen so that multiple grid points fall within one helical period), and the solver evaluates an approximate profile C(r_i) over a selected domain length L. The solver may apply boundary conditions or constraints suitable for the implementation, including fixed-value boundary conditions (e.g., C(0)-1 and C(L)=1), periodic boundary conditions, or natural boundary conditions derived from the Lagrangian formulation. The solver may further apply convergence criteria and numerical tolerances (e.g., a maximum residual threshold, a maximum iteration count, or a mesh-refinement stopping rule) sufficient to produce a stable numerical approximation of C(r) for comparison against the disclosed structural acceptance criterion.

In one expressly disclosed embodiment, the Euler-Lagrange equation derived from the DNA Lagrangian yields a stable-solution form given approximately by:

C(r) approx 1−0.005*cos(2*pi*r/P)

This expression provides one concrete example of an admissible structural profile under the disclosed framework. The profile oscillates weakly about unity and remains close to the idealized stable value across the domain, thereby representing a candidate configuration that preserves structural regularity while reflecting helix-coupled periodicity through the pitch parameter P. The disclosed system may use this expression directly as an analytical reference, may compare a numerically derived profile against this expected form, or may use it as one exemplary benchmark for determining whether a candidate DNA program exhibits acceptable structural stability behavior.

The disclosed framework further defines an explicit acceptance criterion for structural stability. In one embodiment, a candidate is treated as structurally admissible if the base-pairing profile satisfies:

abs(C(r)−1)<=0.05

over the selected evaluation domain. This criterion may be implemented by comparing the computed or approximated stability curve (650) against a stability tolerance band (660) centered on the stable value of unity. Where the profile remains within the disclosed tolerance band, the candidate may be treated as structurally stable for purposes of continuing through the DNARP evaluation method (300). Where the profile deviates outside the tolerance band, the candidate may be rejected, halted, flagged as unstable, or otherwise treated as failing the structural stability evaluation step (350). The use of an explicit tolerance condition is significant because it provides an objective decision rule rather than an informal qualitative judgment. It therefore strengthens determinacy, supports examiner-readiness, and aligns the stability analysis with the disclosed pass/fail structure used elsewhere in the DNARP framework.

The structural stability evaluation is therefore operational rather than merely theoretical. In one implementation, once the candidate DNA program has passed sequence-level and shape-level admissibility checks, the disclosed system retrieves the candidate helical pitch from the shape component (220), instantiates the DNA Lagrangian (610) using the applicable values of kappa_DNA, lambda_DNA, and P, computes or approximates the resulting profile C(r), and then determines whether the resulting stability curve (650) remains inside the stability tolerance band (660). If the tolerance criterion is satisfied, the candidate proceeds to later coherence validation. If the tolerance criterion is not satisfied, the candidate may be rejected before coherence analysis and output prediction are attempted. Thus, the structural stability evaluation acts as a concrete gating stage in the overall DNARP pipeline rather than as a background mathematical observation.

In one aspect, the structural stability evaluation also provides a bridge between the shape component (220) and the later governing constructs. Because the cosine term in the DNA Lagrangian depends explicitly on P, the selected helical pitch affects whether the computed profile remains stable. This means that the shape admissibility range disclosed earlier, such as P in [30, 40] angstrom, is reinforced by the structural stability computation rather than merely duplicated. In one expressly described embodiment, the stability condition is satisfied when P lies within the disclosed range. Thus, the earlier tuple-level shape constraints and the later governing-construct evaluation are technically aligned: the shape component supplies a pitch value, and the structural stability evaluation determines whether that pitch supports an admissible base-pairing profile under the disclosed Lagrangian.

The disclosed structural stability evaluation may also be used comparatively across different candidate DNA programs. For example, two candidates may both satisfy sequence thresholds and nominal geometry ranges, yet differ in how closely their computed C(r) profiles remain within the tolerance band. In such a case, the disclosed system may treat one candidate as more structurally robust than another, may use stability margin as an optimization metric, or may retain only those candidates having a desired stability reserve relative to the tolerance boundary. The governing construct therefore supports not only binary pass/fail rejection, but also structured comparison among candidate designs, provided that the core acceptance rule remains grounded in the disclosed tolerance-based framework.

In some embodiments, the disclosed system may implement the structural stability evaluation using exact or approximate values for kappa_DNA and lambda_DNA, discretized versions of r, or bounded numerical approximations to the solution curve, without departing from the disclosed invention. Likewise, the selected domain for checking abs (C(r)−1)<=0.05 may correspond to a full construct, a representative segment, one or more helical periods, or another domain selected according to implementation needs. The important technical point is that the disclosed system uses a defined structural governing construct, computes or approximates a stability profile from that construct, and applies an explicit acceptance rule tied to a disclosed tolerance band. This preserves the operational core of the structural stability evaluation while permitting standard implementation flexibility in solver choice and computational representation.

Accordingly, the structural stability evaluation described herein provides a concrete, machine-implementable mechanism for determining whether a candidate DNARP program is structurally admissible beyond simple sequence and geometry thresholding. By using the DNA Lagrangian (610), by defining the operative terms C(r), r, P, kappa_DNA, and lambda_DNA, by solving or approximating a corresponding stability profile with the stability solver (640), by comparing the resulting stability curve (650) to the stability tolerance band (660), and by accepting or rejecting the candidate according to the explicit condition abs (C(r)−1)<=0.05, the disclosed system converts structural stability into a deterministic computational stage within the DNARP evaluation method (300). This governing construct therefore forms a central part of the disclosed DNA programming and simulation framework and provides the structural basis on which later coherence validation and output prediction may reliably proceed.

Coherence validation is performed in the disclosed DNARP framework to determine whether the coherence-related state requested by a candidate DNA program is an allowed and admissible state for the candidate configuration. In the present disclosure, coherence validation is not treated as a speculative or purely descriptive assignment of a preferred energetic condition. Rather, it is implemented as a defined computational stage in which the disclosed system applies a DNA-adapted recognition operator to the candidate state, evaluates whether the requested coherence level corresponds to an allowed operator-governed state, and rejects the candidate if the requested state is not admissible. This coherence validation corresponds to the coherence validation step (360) in the DNARP evaluation method (300) of (FIG. 3) and is further illustrated in the coherence validation diagram (700) of (FIG. 7), which includes the normalized coordinate (710), DNA recognition operator (720), eigenvalue solver (730), allowed coherence state set (740), discrete coherence energy level (750), eigenfunction profile (760), and admissibility check (770).

In one disclosed embodiment, the governing construct used for coherence validation is a DNA-adapted recognition operator, referred to herein as H_DNA. The operator may be expressed in ASCII-safe notation as:

H_DNA=−i*x*(d/dx)−i/2+k_DNA*X_DNA*(x+X_DNA*cos(2*pi*x/(P/X_DNA)))

In this expression, x is a normalized coordinate, P is the helical pitch associated with the candidate DNA design, X_DNA is the characteristic DNA recognition length previously described, and k_DNA is an implementation constant used in the operator formulation. The operator H_DNA may be understood as the DNA-specific form of the broader recognition-operator concept introduced earlier in connection with the Recognition Science basis used by the present disclosure. In the DNARP system, however, the operator is not used in the abstract.

Instead, it is used concretely to evaluate whether a candidate-requested coherence-related state is permitted for the candidate DNA configuration under the disclosed framework.

The normalized coordinate x may be defined as:

x=r/X_DNA

where r is the helix-associated coordinate used in the structural framework and X_DNA is the characteristic DNA recognition length. This normalization is useful because it places the coherence evaluation on a dimensionless coordinate basis tied to the common DNA length scale derived from the Recognition Science parameter set. By using x=r/X_DNA, the disclosed operator formulation relates the local position variable to the same underlying scale framework that also supports the preferred helical pitch, the coherence-energy quantum, and the rate relationships elsewhere in the DNARP system. The normalized coordinate (710) shown in (FIG. 7) therefore provides the coordinate basis on which the disclosed operator-based validation is carried out.

The implementation constant k_DNA may be selected, derived, estimated, or otherwise assigned in a manner consistent with the disclosed operator formulation. In one embodiment, k_DNA functions as a coupling or scale-related constant that controls the contribution of the geometry-linked operator term involving x and the cosine function. The present disclosure does not require that every implementation use one exact solver, one exact numeric precision, or one exact parameter-extraction routine for k_DNA, so long as the disclosed system applies the operator formulation in a manner that preserves the technical relationship among x, X_DNA, P, and the operator-governed admissibility test. Thus, k_DNA may be implemented using direct assignment, parameter lookup, calibration within disclosed tolerances, or another appropriate computational technique consistent with the disclosed framework.

The disclosed DNARP framework uses the operator H_DNA to evaluate whether the requested coherence energy level belongs to an allowed state structure. In one expressly disclosed embodiment, the allowed coherence states satisfy:

E_n=n*E_coh

with:
n in {1, 2, 3}

Thus, the candidate does not merely carry an arbitrary energy label. Rather, it requests one of a discrete set of operator-governed coherence states, and the disclosed system determines whether that state is admissible. As shown in (FIG. 7), the discrete coherence energy level (750) may be compared against an allowed coherence state set (740) by the eigenvalue solver (730). In this way, coherence validation is performed as a state-membership and admissibility determination rather than as an unconstrained numeric estimate.

In one implementation, the disclosed system tests whether the requested energy level E_n matches an allowed eigenvalue or allowed discrete operator-associated level for the candidate configuration. If the requested level does not correspond to an allowed state in the disclosed set, the candidate may be rejected at the coherence validation step (360) before final output prediction is performed. This rejection may occur even where the candidate has already satisfied sequence-level admissibility, geometric admissibility, and structural stability conditions.

Accordingly, coherence validation operates as a distinct and independent gating step that must be satisfied before the candidate proceeds to final output computation. This is significant because it ensures that a candidate accepted by the DNARP framework is not merely sequence-valid and structurally stable, but also coherence-valid under the disclosed operator-governed state logic. In addition to testing whether a requested coherence level corresponds to an allowed eigenvalue or allowed discrete state, the disclosed system may also evaluate whether a corresponding state function is admissible. In one expressly described embodiment, the candidate may be associated with an eigenfunction profile approximated by:

psi_n(x) approx x\hat{ }(1/2)*exp(−x/13.6)*cos(n*2*pi*x/35.6)

This expression provides one exemplary state-function form associated with the candidate coherence level. The disclosed system may use such a form as a direct analytical reference, may compare a numerically generated state function against this form, or may use it as a representative admissibility profile for the purpose of determining whether the requested state is normalizable, bounded, computationally well-behaved, or otherwise consistent with the disclosed operator-governed framework. Thus, coherence validation may include not only a check on the requested energy level itself, but also a check on whether the corresponding operator-associated state function is admissible.

The eigenfunction profile (760) shown in (FIG. 7) therefore represents one exemplary way in which the system may assess state admissibility beyond simple numeric level matching. In one embodiment, the admissibility check (770) may determine whether the corresponding state function remains finite over a selected domain, whether it is normalizable, whether it conforms to a required oscillatory structure associated with the selected integer index n, or whether it otherwise satisfies a solver-defined admissibility criterion. If the state function fails the admissibility check, the candidate may be rejected even if the nominal requested energy level equals an allowed multiple of E_coh. This provides a more robust validation logic because it prevents the candidate from passing coherence validation merely by asserting a permitted energy multiple without corresponding state-level support under the operator formulation.

The disclosed coherence validation is therefore operational rather than merely theoretical. In one implementation, after the candidate DNA program has passed sequence-level admissibility, shape-level admissibility, and structural stability evaluation, the system determines the requested coherence level from the energy component (230), computes or identifies the corresponding discrete state E_n, instantiates the operator H_DNA using the applicable values of x, X_DNA, P, and k_DNA, and then determines whether the requested state is admissible according to the disclosed operator-governed structure. This may include testing whether the requested state matches one of the allowed discrete coherence levels, whether a corresponding eigenvalue is present in the allowed set, whether a corresponding state function is admissible, or any combination of those checks. If the candidate passes these checks, the candidate proceeds to output prediction. If the candidate fails, the candidate may be rejected or halted at the coherence validation step (360).

The operator formulation also reinforces the coordinated relationship among the sequence component, shape component, and energy component. Because the operator H_DNA depends on the helical pitch P, the shape component of the candidate DNA program remains relevant during coherence validation. A candidate that selects an otherwise allowed discrete state may nevertheless fail coherence validation if its geometric parameters do not support an admissible operator-governed state under the disclosed formulation. Likewise, as discussed previously, higher requested coherence levels may require stronger sequence-level support. Thus, coherence validation is not isolated from the rest of the tuple. Rather, it is the stage at which the requested coherence state is tested against a formulation that is already conditioned by the candidate's DNA-derived parameters and geometry. This preserves the integrated character of the DNARP framework and prevents coherence selection from becoming detached from the physical and structural support encoded elsewhere in the candidate tuple.

In some embodiments, the disclosed system may use exact analytical solving, numerical approximation, discretized operator representations, matrix-based eigensolvers, iterative methods, spectral methods, or equivalent computational techniques to determine whether a requested state is admissible. The domain over which the state function is evaluated may correspond to a selected normalized interval, a representative structural region, one or more helical periods, or another computational domain suitable for coherence-state assessment. The present disclosure does not require one exclusive solver architecture. The important technical point is that the system uses a defined DNA-adapted recognition operator, applies that operator to the candidate state, evaluates whether the requested coherence level belongs to the allowed state structure, and rejects the candidate if the requested state is invalid or non-admissible.

The coherence validation diagram (700) shown in (FIG. 7) illustrates one exemplary implementation of these operations. The normalized coordinate (710) supplies the dimensionless coordinate basis. The DNA recognition operator (720) provides the governing operator construct.

The eigenvalue solver (730) evaluates the candidate-requested state against the allowed coherence state set (740) and the discrete coherence energy level (750). The eigenfunction profile (760) represents a corresponding state-function form or approximation, and the admissibility check (770) determines whether the candidate passes the coherence-validation requirements. Although (FIG. 7) illustrates one representative arrangement of these elements, the disclosed invention is not limited to that exact internal module arrangement. Equivalent operator-evaluation engines, eigenvalue-checking routines, normalization modules, or admissibility-testing procedures may be used so long as they implement the disclosed technical logic of validating a candidate's requested coherence state through an operator-based computational framework.

Accordingly, the coherence validation described herein provides a concrete, machine-implementable mechanism for determining whether a candidate DNARP program requests an allowed and admissible coherence state. By using the DNA recognition operator (720), by defining the normalized coordinate x=r/X_DNA, by evaluating the requested state against the allowed coherence state set (740), by using the discrete coherence energy level (750) as an operator-governed state quantity, by optionally assessing an associated eigenfunction profile (760), and by performing an admissibility check (770) that accepts or rejects the candidate, the disclosed system converts coherence-state selection into a deterministic computational stage within the DNARP evaluation method (300). This governing construct therefore forms a central part of the disclosed DNA programming and simulation framework and provides the coherence-state basis on which final predicted functional output may be computed.

Once a candidate DNARP program has satisfied sequence-level admissibility, shape-level admissibility, structural stability evaluation, and coherence validation, the disclosed system computes a predicted functional output for that candidate. In the present disclosure, output prediction is not treated as an informal estimate appended after the principal validation stages. Rather, it is a defined computational stage in which the system applies a recognition transform to the validated candidate state, determines an output amplitude associated with the candidate program, and then combines that output amplitude with the candidate's associated rate to generate a predicted functional output. This stage corresponds to the predicted output computation step (370) and candidate output step (380) of the DNARP evaluation method (300) shown in (FIG. 3), and is further illustrated in the output prediction diagram (800) of (FIG. 8), which includes the recognition transform (810) and the predicted functional output (820).

In one disclosed embodiment, the governing construct used for output prediction is a Recognition Transform denoted F_DNA(E_n). The transform is defined in terms of the sequence stability score, the selected coherence level, and the candidate helical geometry. An exemplary ASCII-safe form of the transform is:

Gamma_norm(z)=Gamma(z)/abs(Gamma(z))

F_DNA(E_n)=(S_Stab/3)*Gamma_Norm(0.5+i*E_n/E_Coh)*Exp(i*2*Pi*P/P_0)

In this formulation, S_stab is the sequence stability score obtained from the sequence component, E_n is the validated coherence-energy level obtained from the energy component, E_coh is the coherence-energy quantum previously defined in the RS-derived DNA parameter set, P is the helical pitch carried by the shape component, and P_0 is the preferred helical pitch derived from the common Recognition Science scaling framework. The factor Gamma_norm(z) is a normalized gamma-function phase term defined so that its magnitude is unity. Accordingly, the transform combines a stability-derived amplitude scale, a coherence-related phase term, and a geometry-related phase term into a single complex-valued output-prediction construct.

The normalized gamma term Gamma_norm(z) is used in the present disclosure to preserve a phase-sensitive transform structure while maintaining a controlled magnitude contribution.

Because Gamma_norm(z) is defined as Gamma(z)/abs(Gamma(z)), its absolute value is equal to one, and it therefore contributes phase information without altering the transform magnitude.

Likewise, the exponential term exp (i*2*pi*P/P_0) is a unit-magnitude phase factor tied to the ratio of the candidate helical pitch P to the preferred helical pitch P_0. As a result, the magnitude of the transform is governed principally by the sequence-dependent prefactor (S_stab/3), while the remaining terms preserve coherence-related and geometry-related phase structure. This is useful because it allows the output amplitude to depend monotonically and directly on the disclosed sequence stability formulation while still retaining explicit dependence on the candidate's validated coherence level and helix geometry within the transform representation.

In one expressly disclosed embodiment, the output amplitude squared associated with the Recognition Transform is given by:

abs(F_DNA(E_n))\hat{ }2=(S_stab/3)\hat{ }2

This expression follows because the normalized gamma term and the exponential term each have unit magnitude in the disclosed formulation. The transform magnitude therefore scales directly with the normalized sequence stability score. This relationship is significant because it makes the output amplitude a deterministic function of S_stab under the disclosed framework. A candidate having a higher sequence stability score will produce a larger transform magnitude than a candidate having a lower sequence stability score, all else being equal. Stated differently, the disclosed output amplitude is monotone increasing in S_stab, which provides a direct and interpretable relationship between sequence-level support and output-prediction strength.

The disclosed system then combines the transform amplitude with the candidate's associated rate R to compute a final predicted functional output. In one embodiment, the final output is defined as:

Output(D)=abs(F_DNA(E_n))\hat{ }2*R

Substituting the disclosed amplitude relationship and the previously defined rate relationship R=n*R_0, the final output may be written as:

Output(D)=(S_stab/3)\hat{ }2*n*R_0

This expression provides a compact and deterministic prediction rule for the candidate DNARP program D. The predicted output therefore depends on two principal validated quantities: first, the sequence-derived amplitude factor (S_stab/3)\hat{ }2, and second, the coherence-state-derived rate factor n*R_0. Because both quantities arise only after the candidate has satisfied the earlier validation stages of the DNARP pipeline, the predicted output is not a free-floating estimate.

Rather, it is the result of applying the disclosed Recognition Transform and rate logic to a candidate that has already passed sequence, geometry, structural, and coherence admissibility checks.

The transform-based output formulation is useful because it provides a common computational endpoint for the unified tuple representation D=(S, H, E). The sequence component contributes the sequence stability score S_stab. The shape component contributes the helical pitch P, which appears in the transform phase factor and is also constrained by the earlier admissibility and structural stability stages. The energy component contributes the validated coherence level E_n and the associated rate R. Thus, the final predicted functional output is not derived from only one component of the tuple. Instead, it is the output-stage consequence of the full DNARP representation after that representation has been processed through the disclosed validation framework. This preserves the integrated character of the invention and reinforces that the disclosed system is a complete DNA programming and evaluation framework rather than a sequence-only scoring tool or a separate rate-prediction utility.

In one aspect, the disclosed transform structure provides an interpretable relationship between baseline candidates and enhanced candidates. Because the output amplitude squared is proportional to (S_stab/3)\hat{ }2, a baseline candidate satisfying the minimum admissible stability threshold may be used as a reference point for comparing candidates having stronger sequence support. Likewise, because the rate term scales as n*R_0, a candidate evaluated at a higher validated coherence level may produce a correspondingly higher predicted output than a candidate evaluated at a lower coherence level, provided that both candidates are otherwise admissible. This enables the disclosed system to express output improvement in a structured way. For example, a candidate may be described as having a fold enhancement relative to a baseline candidate or baseline state by comparing the candidate's transform-derived amplitude, or by comparing final output values under different valid settings of S_stab and n.

In one embodiment, fold enhancement may be computed relative to a selected baseline state by comparing (S_stab/3)\hat{ }2 for a candidate against the corresponding baseline amplitude value. In another embodiment, fold enhancement may be computed using the full output expression, such as by comparing Output (D) for a candidate against the output associated with a baseline coherence level or baseline sequence stability score. The present disclosure does not require one exclusive fold-enhancement reporting convention so long as the comparison remains grounded in the disclosed transform and output relationships. What is important is that the disclosed framework permits such comparisons to be made deterministically from validated tuple parameters rather than from external empirical fitting. Thus, fold enhancement is a permissible derivative quantity of the output-prediction stage, although the core computation remains the predicted functional output itself.

The output-prediction stage is therefore operational rather than merely illustrative. In one implementation, after a candidate DNA program has passed the coherence validation step (360), the system retrieves the sequence stability score S_stab, the validated coherence level E_n, the associated rate R, and the candidate helical pitch P, then applies the recognition transform (810) of the output prediction diagram (800) to determine the transform magnitude and compute the predicted functional output (820). The resulting output may then be provided at the candidate output step (380) as a numerical prediction, a comparative value, a baseline-relative metric, or another machine-generated output form consistent with the disclosed framework. If desired, the system may also store the output value, compare it against target criteria in later optimization stages, or use it to rank candidate designs, but the core technical operation remains the disclosed transform-based computation of predicted functional output.

In the presently disclosed embodiment, the output-prediction stage also preserves determinacy by relying only on previously validated inputs. The sequence stability score has already been computed and checked for admissibility. The helical pitch has already been screened and used in structural stability analysis. The coherence level has already been validated against the DNA recognition operator. The associated rate has already been tied to the discrete coherence state through R=n*R_0. As a result, when the system computes Output (D)=(S_stab/3)\hat{ }2*n*R_0, it is not extrapolating from unconstrained parameters. It is combining a set of quantities that have already been constrained by the disclosed DNARP framework. This sequence of operations improves technical coherence and provides a deterministic description of how the system arrives at a final predicted output from a candidate DNA program.

The output prediction diagram (800) shown in (FIG. 8) illustrates one exemplary implementation of these operations. The recognition transform (810) represents the transform stage that acts on the validated candidate state, and the predicted functional output (820) represents the resulting machine-generated output value associated with the candidate DNA program. Although (FIG. 8) illustrates a lean output-prediction arrangement, the disclosed invention is not limited to any single software architecture, internal data representation, or display format for carrying out the transform-based computation. Equivalent transform-evaluation routines, complex-phase handling modules, numerical computation engines, or output-generation modules may be used so long as they implement the disclosed technical logic of computing a transform-derived output amplitude and combining that amplitude with the validated rate quantity to generate a predicted functional output.

Accordingly, the output prediction described herein provides a concrete, machine-implementable mechanism for determining a predicted functional output for a candidate DNARP program after the candidate has passed the preceding validation stages of the disclosed framework. By defining the normalized gamma term Gamma_norm(z), by defining the recognition transform F_DNA (E_n), by establishing that abs(F_DNA(E_n))\hat{ }2=(S_stab/3)\hat{ }2, and by computing the final output according to Output(D)=abs(F_DNA (E_n))\hat{ }2*R=(S_stab/3)\hat{ }2*n*R_0, the disclosed system converts validated sequence, geometry, and coherence information into a deterministic output value that is directly tied to the formal DNARP tuple and the preceding simulator stages. This output-prediction construct therefore forms the final computational stage of the core DNARP validation-and-prediction pipeline and provides the technical basis for later candidate acceptance, comparison, tuning, and application-specific use.

The disclosed DNARP evaluation method (300) provides a computer-implemented workflow by which a candidate DNA program is received, parsed, validated, analyzed, and used to generate a predicted functional output. In the present disclosure, the DNARP evaluation method is not an abstract description of how one might generally reason about a DNA design. Rather, it is a defined sequence of computational operations arranged to process a candidate DNARP program in a deterministic manner using the sequence component, shape component, and energy component of the tuple representation. As shown in (FIG. 3), the DNARP evaluation method (300) includes a candidate program receipt step (310), a tuple parsing step (320), a sequence stability computation step (330), a shape constraint validation step (340), a structural stability evaluation step (350), a coherence validation step (360), a predicted output computation step (370), and a candidate output step (380). These steps collectively provide one concrete way to implement the disclosed DNA programming and simulation framework.

In one embodiment, the method begins by receiving a candidate DNARP program at the candidate program receipt step (310). The candidate may be received through a user interface, an application programming interface, a stored candidate library, a file import operation, a design-generation routine, or another input mechanism capable of supplying a DNARP program definition (120) to the DNARP design and simulation system (100) shown in (FIG. 1). The received candidate program may already be expressed as a tuple D=(S, H, E), or may be provided in another structured form that can be converted into that tuple representation by the system. In this respect, the candidate program receipt step (310) is not limited to a single data-entry format. Its function is to place the candidate DNA design into the disclosed evaluation pipeline in a form that allows the later validation and prediction stages to operate on a consistent program structure.

After receipt of the candidate program, the disclosed method proceeds to the tuple parsing step (320). At this stage, the system interprets the incoming candidate definition and separates or maps the candidate into its sequence component, shape component, and energy component.

Where the candidate is already stored in tuple form, the parsing operation may involve extracting the component fields and loading them into internal data structures used by the simulator. Where the candidate is supplied in a different but compatible format, the tuple parsing step (320) may involve normalization, conversion, field mapping, validation of required fields, or other preprocessing sufficient to obtain a machine-usable DNARP program tuple. In either case, the tuple parsing step (320) provides the operative bridge between candidate definition and candidate evaluation by placing the program into the form required for the later computational stages.

In one embodiment, once the tuple is parsed, the method proceeds to the sequence stability computation step (330). At this stage, the system operates on the sequence component of the candidate program and computes the sequence stability score S_stab according to the disclosed sequence formulation. As discussed previously, the system may count CG pairs and AT pairs, apply the weighted sequence-stability relation, and determine whether the candidate satisfies the required sequence threshold for a valid DNARP program. In one expressly disclosed embodiment, the system computes:

S_stab=1.5*(number of CG pairs)+1.0*(number of AT pairs)

and then determines whether the candidate satisfies the required threshold condition:

S_stab>=3

or, in implementations using functional units, S_stab>=3 per functional unit. This step may be performed using the sequence evaluation subsystem (400) shown in (FIG. 4), including the codon sequence input (410), CG pair count (420), AT pair count (430), stability scoring engine (440), sequence stability score (450), sequence validity threshold (460), and threshold comparator (470). If the sequence stability score fails the applicable threshold, the method may halt and reject the candidate before later geometry, structural, coherence, and output operations are performed.

In one embodiment, after sequence stability has been computed and found sufficient, the method proceeds to the shape constraint validation step (340). At this stage, the system evaluates the shape component of the candidate DNARP program, including at least the helical pitch P and groove ratio G, to determine whether the candidate satisfies the disclosed geometric admissibility conditions. In one expressly disclosed embodiment, the system checks whether:
P in [30, 40] angstrom
and
G in [1.5, 1.7]

The disclosed shape constraint validation step (340) may be performed using the shape evaluation subsystem (500) shown in (FIG. 5), including the DNA helix (510), helical pitch (520), major groove (530), minor groove (540), groove ratio (550), permitted pitch range (560), and permitted groove-ratio range (570). If the candidate pitch or groove ratio lies outside the permitted range, the candidate may be rejected at this stage. Accordingly, the method ensures that a candidate is not advanced to later structural and coherence analysis unless the candidate first satisfies the disclosed geometric constraints.

In one embodiment, the method also evaluates the energy component during the early validation stage to determine whether the requested coherence-related state and associated rate conform to the disclosed discrete-state logic. This check may occur as part of the tuple parsing step (320), as part of the sequence stability computation step (330), as part of the shape constraint validation step (340), or as a separate internal validation operation preceding the structural stability evaluation step (350). In one expressly disclosed embodiment, the system checks whether the requested coherence state satisfies:

E_n=n*E_coh

with:
n in {1, 2, 3}
and whether the associated rate satisfies:
R=n*R_0

Thus, the system may determine at an early stage whether the candidate requests an allowed coherence level and a rate consistent with that level. If the candidate requests a coherence index outside the allowed set or a rate inconsistent with the requested index, the candidate may be rejected before the more detailed operator-based coherence analysis is performed. This early check improves computational efficiency and reinforces that the energy component is part of the candidate program definition itself rather than merely a downstream output label.

In one embodiment, once the candidate has passed the early sequence, shape, and basic energy checks, the method proceeds to the structural stability evaluation step (350). At this stage, the system instantiates the DNA Lagrangian using the candidate pitch and applicable implementation parameters, solves or approximates the corresponding Euler-Lagrange equation, computes a stability profile C(r) across a selected domain, and determines whether the candidate satisfies the disclosed stability criterion. In one expressly disclosed embodiment, the system evaluates whether:

abs(C(r)−1)<=0.05

over the selected domain, such that the resulting stability curve remains within a disclosed tolerance band around the stable value. This operation may be performed using the structural stability evaluation diagram (600) shown in (FIG. 6), including the DNA Lagrangian (610), coordinate variable (630), stability solver (640), stability curve (650), and stability tolerance band (660). If the candidate fails the structural stability criterion, the method may halt and report the candidate as structurally unstable. Thus, the structural stability evaluation step (350) provides a concrete gating stage between early syntactic or threshold validation and later coherence-based validation.

In one embodiment, after structural stability has been confirmed, the method proceeds to the coherence validation step (360). At this stage, the system uses the DNA recognition operator (720) to determine whether the candidate-requested coherence state is an allowed and admissible state for the candidate configuration. As discussed previously, this may include determining whether the discrete coherence energy level corresponds to an allowed state in the allowed coherence state set (740), whether a corresponding eigenvalue is present, and whether a corresponding eigenfunction profile is admissible under the disclosed operator framework. The coherence validation step (360) may be performed using the coherence validation diagram (700) shown in (FIG. 7), including the normalized coordinate (710), DNA recognition operator (720), eigenvalue solver (730), discrete coherence energy level (750), eigenfunction profile (760), and admissibility check (770). If the candidate fails the coherence validation step (360), for example because the requested state is invalid or because the corresponding state function is not admissible, the method may halt and reject the candidate before any final predicted output is generated.

In one embodiment, once the candidate has passed sequence-level admissibility, shape-level admissibility, structural stability evaluation, and coherence validation, the method proceeds to the predicted output computation step (370). At this stage, the system computes the predicted functional output for the candidate program using the validated tuple parameters and the disclosed recognition transform. More particularly, the system may compute the transform magnitude according to the disclosed F_DNA(E_n) relation, determine that abs(F_DNA (E_n))\hat{ }2=(S_stab/3)\hat{ }2, and then compute the predicted output according to:

Output(D)=abs(F_DNA(E_n))\hat{ }2*R

or equivalently:

Output(D)=(S_stab/3)\hat{ }2*n*R_0

This stage may be carried out using the output prediction diagram (800) shown in (FIG. 8), including the recognition transform (810) and predicted functional output (820). In this way, the predicted output computation step (370) converts the validated sequence, geometry, and coherence-state information into a final machine-generated output value tied directly to the candidate DNARP program.

In one embodiment, after the predicted functional output has been computed, the method proceeds to the candidate output step (380). At this stage, the system may provide a result indicating whether the candidate has passed the disclosed evaluation process and, if so, what predicted functional output is associated with the candidate. The output may include one or more result categories such as stability status, coherence-validity status, predicted functional output, baseline-relative enhancement, or another result format consistent with the disclosed framework.

Where the candidate has failed at an earlier stage, the candidate output step (380) may provide an error condition, rejection status, or failure classification indicating the stage at which the candidate ceased to qualify. Thus, the candidate output step (380) serves not merely as a display stage, but as the formal conclusion of the evaluation pipeline in which the system commits to an acceptance, rejection, or classified result based on the disclosed decision logic.

In one expressly disclosed implementation, the method may be understood as applying a deterministic pass/fail sequence of decision rules. For example, the system may reject the candidate if S_stab<3, reject the candidate if P lies outside [30, 40] angstrom, reject the candidate if G lies outside [1.5, 1.7], reject the candidate if the requested coherence index is not in {1, 2, 3}, reject the candidate if the associated rate does not satisfy R=n*R_0, reject the candidate if the structural stability tolerance criterion fails, and reject the candidate if coherence validation fails. Only if all such required checks are satisfied does the system compute and report the predicted functional output for the candidate. This arrangement is significant because it makes the DNARP evaluation method (300) a deterministic acceptance-and-prediction workflow rather than a vague modeling pipeline. Each stage has an objective role, and the method as a whole yields a defined result based on disclosed thresholds, ranges, equations, and admissibility criteria.

In one embodiment, the output module outputs a validity record comprising: (i) a sequence-valid flag, (ii) a shape-valid flag, (iii) a stability-valid flag, (iv) a coherence-valid flag, and (v) when all flags are true, a predicted functional output value. In one embodiment, the candidate is rejected when any flag is false and accepted when all flags are true.

The DNARP evaluation method (300) may also be understood in relation to the module-level architecture of the DNARP design and simulation system (100) shown in (FIG. 1). In one implementation, the candidate program receipt step (310) and tuple parsing step (320) may be supported by the input validation module (130), the structural stability evaluation step (350) may be supported by the stability analysis module (140), the coherence validation step (360) may be supported by the coherence validation module (150), the predicted output computation step (370) may be supported by the function prediction module (160), and the candidate output step (380) may be supported by the output module (170). Thus, the method illustrated in (FIG. 3) and the system architecture illustrated in (FIG. 1) may be viewed as complementary views of the same disclosed invention, with the former emphasizing ordered operations and the latter emphasizing functional implementation modules.

In some embodiments, the disclosed method may optionally include an iterative refinement loop after the candidate output step (380) or after one or more earlier rejection conditions. For example, if a candidate fails the sequence threshold, the system may modify sequence content and re-enter the method at the tuple parsing step (320) or sequence stability computation step (330). If a candidate satisfies the admissibility requirements but does not meet a target predicted output, the system may alter the sequence component, adjust the target coherence level within the allowed set, or select a different geometric configuration and then re-evaluate the modified candidate. Such iteration does not change the underlying DNARP evaluation method. Rather, it uses repeated application of the same disclosed validation-and-prediction workflow to explore candidate designs until an acceptable output or design objective is achieved. This optional iterative use is consistent with the disclosed system's support for candidate refinement, regulatory tuning, therapeutic design selection, and other application-driven workflows.

The disclosed DNARP evaluation method (300) is therefore one central operational expression of the present invention. By receiving a candidate program at the candidate program receipt step (310), parsing the tuple at the tuple parsing step (320), computing sequence stability at the sequence stability computation step (330), validating geometric constraints at the shape constraint validation step (340), evaluating structural stability at the structural stability evaluation step (350), validating coherence at the coherence validation step (360), computing predicted functional output at the predicted output computation step (370), and reporting the candidate result at the candidate output step (380), the disclosed method provides a concrete and fully machine-implementable path for operating on a candidate DNA program represented in the DNARP language. This method ties together the sequence, shape, energy, stability, coherence, and output constructs previously described into a single deterministic computational workflow for evaluating a candidate DNA program.

In one fully implementable embodiment, the disclosed DNARP framework is used to design and evaluate a candidate promoter or coding-region DNA molecule by receiving a defined set of candidate inputs, computing sequence, shape, and energy quantities for the candidate, applying the disclosed validation logic in an ordered manner, and outputting a deterministic acceptance, rejection, or refinement result. This embodiment is computer-implemented and may be carried out by the DNARP design and simulation system (100) using the DNARP evaluation method (300) shown in (FIG. 3), together with the sequence evaluation subsystem (400) of (FIG. 4), the shape evaluation subsystem (500) of (FIG. 5), the structural stability evaluation diagram (600) of (FIG. 6), the coherence validation diagram (700) of (FIG. 7), and the output prediction diagram (800) of (FIG. 8). The embodiment is presented as one concrete way to implement the disclosed invention and is structured so that a skilled person may reproduce the evaluation flow using the disclosed parameters, equations, thresholds, and decision rules.

In one implementation, the embodiment begins with definition of a candidate DNARP program having a sequence input, a geometry input, and a coherence-state input. The candidate sequence input may be supplied as a codon list, nucleotide string, grouped sequence block representation, or another machine-usable sequence form. In one embodiment directed to a promoter or coding-region candidate, the sequence input is provided as a candidate codon list constituting the sequence component of the candidate tuple. The geometry input includes a candidate helical pitch and a candidate groove ratio corresponding to the shape component of the tuple. The coherence-state input includes a target coherence level n, and in some implementations may additionally include a target output or target fold-enhancement value against which the candidate will be assessed. For example, a user may request evaluation of a candidate coding-region sequence at a target coherence level n=2, or may request design of a regulatory candidate expected to achieve at least a selected fold enhancement relative to a baseline state. These values may be received through the user or API input (110) and stored as part of the DNARP program definition (120) before the candidate is processed through the input validation module (130).

After the candidate inputs are received, the system determines or retrieves the theory-derived parameters needed to evaluate the candidate. In one embodiment, the system determines or looks up the quantities E_coh, R_0, P_0, and G_0, and may further determine implementation constants such as kappa_DNA, lambda_DNA, and k_DNA for use in the structural stability and coherence computations. These quantities may be computed directly from the disclosed parameter relationships, retrieved from a stored parameter table, loaded from memory as default implementation values, or supplied as precomputed constants for the selected embodiment. In one implementation, the system uses the previously disclosed RS-derived parameter set, including P_0=X_DNA*(phi\hat{ }2), G_0=phi, E_coh=E_Planck*(X_opt)\hat{ }(101), and R_0=(E_coh/h)*8*(phi\hat{ }(−60)), to initialize the evaluation environment for the candidate. This step is useful because it ensures that the candidate is not evaluated against arbitrary or changing parameter origins, but rather against the common parameter basis disclosed for the DNARP framework.

The system then proceeds to process the candidate sequence using the sequence evaluation subsystem (400) shown in (FIG. 4). In one implementation, the candidate sequence is normalized into a machine-usable representation, parsed into codons or equivalent sequence units, and examined to determine a CG pair count (420) and an AT pair count (430). The stability scoring engine (440) then computes the sequence stability score (450) according to the disclosed weighted relation:

S_stab=1.5*(number of CG pairs)+1.0*(number of AT pairs)

The resulting value is compared by the threshold comparator (470) against the sequence validity threshold (460). In one embodiment, the minimum admissibility condition is:

S_stab>=3

or, where the candidate is segmented into functional units, S_stab>=3 per functional unit. If the candidate sequence does not satisfy the threshold, the system rejects the candidate and may output a rejection result indicating that the candidate failed the sequence-level requirement. In one implementation, a candidate coding-region sequence having insufficient CG-supported stability may therefore be halted before any structural or coherence-based calculations are performed. This early rejection conserves computation and ensures that only candidates having adequate sequence-level support proceed further in the pipeline.

If the candidate passes sequence-level screening, the system then processes the candidate geometry using the shape evaluation subsystem (500) shown in (FIG. 5). In one implementation, the candidate helical pitch is treated as the helical pitch (520) of the candidate DNA helix (510), and the candidate groove ratio is treated as the groove ratio (550) defined with respect to the major groove (530) and minor groove (540). The system compares the candidate pitch against the permitted pitch range (560) and compares the candidate groove ratio against the permitted groove-ratio range (570). In one expressly disclosed embodiment, the system checks whether:
P in [30, 40] angstrom
and
G in [1.5, 1.7]

If the candidate geometry lies outside either disclosed range, the system rejects the candidate as geometrically non-admissible. In some embodiments, the system also computes the distance of the candidate geometry from the preferred values P_0 and G_0, and may store or use those distances as compatibility measures, especially where the candidate seeks a higher coherence level or higher predicted output. Thus, this geometry-processing stage may function both as a binary admissibility gate and as a source of later optimization information.

Once the candidate has passed the basic sequence and geometry gates, the system evaluates the candidate's requested coherence state at an initial consistency level. In one implementation, the system receives the target coherence level n as part of the candidate program definition and computes the corresponding candidate coherence energy level and associated rate according to:

E_n=n*E_coh

and

R=n*R_0

In the expressly described embodiment, the allowed coherence states satisfy:
n in {1, 2, 3}

Accordingly, the system verifies whether the requested n belongs to the allowed state set and whether the candidate's associated energy and rate values match the disclosed discrete-state relationships. In one embodiment, if the candidate requests a target coherence level n=2, the system computes E_2=2*E_coh and R=2*R_0, and rejects the candidate if the candidate-supplied or candidate-derived values are inconsistent with that state. Likewise, if the candidate requests a target coherence level not in the allowed set, the system rejects the candidate as failing the energy-component consistency requirement before advancing to the more detailed operator-based coherence validation. This early check allows the system to ensure that the requested state is at least formally well-formed before additional stability and operator computations are undertaken.

The system then performs structural stability computation using the structural stability evaluation diagram (600) shown in (FIG. 6). In one implementation, the stability analysis module (140) instantiates the DNA Lagrangian (610) for the candidate using the candidate pitch and the applicable implementation constants, and then uses the stability solver (640) to solve or approximate a stability profile across the coordinate variable (630). In one disclosed embodiment, the governing stability relation is based on:

L_DNA=(kappa_DNA/2)*(dC/dr)\hat{ }2−lambda_DNA*((1−C\hat{ }2)/2−0.1*C*cos(2*pi*r/P))

where C(r) is the computed base-pairing or structural support profile for the candidate. In one implementation, the solver computes a stability curve (650) and tests whether that curve remains inside the stability tolerance band (660) according to the explicit criterion:

abs(C(r)−1)<=0.05

over a selected domain. A candidate that satisfies this criterion is treated as structurally stable for purposes of further evaluation. A candidate that violates this criterion is rejected as structurally unstable. In one embodiment, the system may use the disclosed stable-solution form C(r) approx 1−0.005*cos (2*pi*r/P) as a direct reference or comparison profile when assessing whether the candidate remains within the required tolerance band.

If the candidate passes the structural stability stage, the system then performs coherence validation using the coherence validation diagram (700) shown in (FIG. 7). In one implementation, the coherence validation module (150) determines the normalized coordinate (710) according to x=r/X_DNA, instantiates the DNA recognition operator (720), and uses the eigenvalue solver (730) to determine whether the candidate-requested coherence level corresponds to an allowed state within the allowed coherence state set (740). In one disclosed embodiment, the operator may be written as:

H_DNA=−i*x*(d/dx)−i/2+k_DNA*X_DNA*(x+X_DNA*cos(2*pi*x/(P/X_DNA)))

The system then evaluates whether the requested discrete coherence energy level (750) matches an allowed operator-associated level and, in some embodiments, whether a corresponding eigenfunction profile (760) is admissible under the disclosed framework. An exemplary state-function form that may be used in this stage is:

psi_n(x) approx x\hat{ }(1/2)*exp(−x/13.6)*cos(n*2*pi*x/35.6)

The admissibility check (770) may determine whether the candidate state is normalizable, bounded, computationally well-behaved, or otherwise acceptable under the selected coherence model. If the candidate fails this coherence validation, the system rejects the candidate even if the candidate previously satisfied the sequence, shape, and structural-stability requirements.

Once the candidate has passed the sequence, geometry, structural stability, and coherence stages, the system computes the candidate's predicted functional output using the output prediction diagram (800) shown in (FIG. 8). In one implementation, the function prediction module (160) applies the recognition transform (810) according to:

Gamma_norm(z)=Gamma(z)/abs(Gamma(z))

F_DNA(E_n)=(S_stab/3)*Gamma_norm(0.5+i*E_n/E_coh)*exp(i*2*pi*P/P_0)

The system then determines the transform magnitude according to:

abs(F_DNA(E_n))\hat{ }2=(S_stab/3)\hat{ }2

and computes the predicted functional output (820) according to:

Output(D)=abs(F_DNA(E_n))\hat{ }2*R

or equivalently:

Output(D)=(S_stab/3)\hat{ }2*n*R_0

This output computation provides a deterministic numerical value tied directly to the validated sequence stability score, the validated coherence level, the associated rate, and the candidate helical geometry. In one implementation, the output value may be reported as a predicted expression-related output, a predicted functional output amplitude, or a baseline-relative enhancement quantity derived from the same transform-based computation.

In one concrete use case, the candidate is a promoter or coding-region DNA molecule specified with a candidate codon list, a candidate helical pitch, a candidate groove ratio, and a target coherence level n=2. The system computes the sequence stability score from the codon list, verifies that the score is at least 3, verifies that P and G lie within the disclosed admissibility ranges, computes E_2=2*E_coh and R=2*R_0, verifies that the candidate is structurally stable under the DNA Lagrangian, verifies that the candidate coherence state is admissible under the DNA recognition operator, and then computes the predicted functional output according to the disclosed transform. If all checks pass, the system marks the candidate as valid and outputs the predicted functional result. If any check fails, the system marks the candidate as invalid and may identify the specific stage at which failure occurred, such as sequence threshold failure, shape-range failure, stability-tolerance failure, or coherence-state failure.

In one embodiment, the system may additionally compare the computed output against a target output or target fold-enhancement value supplied as part of the initial candidate definition. For example, a designer may specify a target fold enhancement T_target relative to a baseline candidate. After computing Output(D) and, where appropriate, computing a corresponding enhancement factor, the system may determine whether the target has been met. If the target is met and all validation checks have passed, the candidate is accepted. If the target is not met, the candidate may still be marked as valid but sub-target, or may be routed into an optimization flow for further refinement. This adds a design-oriented decision layer on top of the core validity determination while preserving the same disclosed computational logic.

In one implementation, the decision rule for the embodiment may therefore be stated explicitly as follows. The system accepts the candidate if: the sequence stability score satisfies the required threshold; the candidate helical pitch lies within the disclosed pitch range; the candidate groove ratio lies within the disclosed groove-ratio range; the requested coherence level belongs to the allowed discrete state set; the structural stability profile satisfies the disclosed tolerance criterion; the coherence state is admissible under the DNA recognition operator; and, where a target output has been specified, the computed predicted output meets or exceeds the target value. The system rejects the candidate if any required validity condition fails. In some embodiments, the system may output a pass/fail result together with the predicted output value and one or more failure reasons or compatibility measurements.

The disclosed embodiment may also include an optional optimization loop. In one implementation, if the candidate fails the sequence threshold or fails to achieve the target output, the system alters one or more codons or sequence units, recomputes the sequence stability score, and re-enters the evaluation flow beginning at the sequence processing stage or at an earlier tuple parsing stage. In another implementation, if the candidate is sequence-valid but not output-optimal, the system may preserve the geometry and target coherence level while altering the sequence component to increase S_stab or to move the candidate more closely toward the target enhancement. In still another implementation, the system may preserve the sequence but explore allowed changes in the requested coherence level or geometric parameters within the disclosed admissibility limits. The optimization loop may terminate when the target output is achieved, when no additional improving modification is available, when an iteration limit is reached, or when another selected termination rule is satisfied.

This fully implementable embodiment therefore provides a concrete recipe by which a promoter or coding-region DNA candidate may be represented, evaluated, accepted, rejected, and optionally refined using the disclosed DNARP framework. By receiving a candidate input set, determining the applicable RS-derived parameters, computing sequence stability through the sequence evaluation subsystem (400), evaluating geometry through the shape evaluation subsystem (500), computing structural stability through the structural stability evaluation diagram (600), validating coherence through the coherence validation diagram (700), computing predicted functional output through the output prediction diagram (800), and applying explicit acceptance and rejection rules tied to disclosed thresholds and criteria, the system provides a reproducible and machine-implementable path for practicing the invention.

The disclosed DNARP framework may be implemented as a computer-based system configured to receive a candidate DNA program, process the program according to the disclosed sequence, shape, structural stability, coherence, and output formulations, and provide one or more resulting validity determinations or predicted functional measures. In one embodiment, this system is represented by the DNARP design and simulation system (100) shown in (FIG. 1), which provides a functional view of the principal processing modules used to evaluate a candidate DNA program, and by the computing environment (1200) shown in (FIG. 12), which provides a hardware and software support view of one suitable implementation platform. These figures may be understood together as illustrating both the logical architecture of the disclosed DNARP framework and one or more practical computing arrangements by which that framework may be implemented.

As shown in (FIG. 1), the DNARP design and simulation system (100) may receive a user or API input (110) and a DNARP program definition (120). The user or API input (110) may include any suitable mechanism by which a candidate DNA design, candidate tuple, target parameter set, target output criterion, candidate library entry, optimization request, or other design-related information is supplied to the system. In one embodiment, the user or API input (110) includes an interactive user interface through which a designer enters a sequence component, shape component, and energy component corresponding to a candidate DNARP program. In another embodiment, the user or API input (110) includes an application programming interface through which an external software application, automated design engine, candidate database, laboratory information system, or remote service submits a candidate DNA program for evaluation. In still other embodiments, the user or API input (110) may include batch-uploaded candidate files, automatically generated design sets, cloud-based requests, or other structured data-transfer mechanisms. The disclosed invention is not limited to any one input modality so long as the system is configured to receive information sufficient to define or derive a candidate DNARP program.

The DNARP program definition (120) may represent the candidate design after entry, normalization, or structuring into a machine-usable form suitable for the disclosed evaluation pipeline. In one embodiment, the DNARP program definition (120) includes a tuple representation D=(S, H, E) having a sequence component, a shape component, and an energy component. In another embodiment, the DNARP program definition (120) is initially received in another structured representation, and the system converts that representation into the tuple form used by the disclosed simulator. The DNARP program definition (120) may therefore function as the internal candidate object on which the disclosed modules operate. In some implementations, the DNARP program definition (120) may also include associated metadata, such as a candidate identifier, an application context, a target output criterion, a target fold-enhancement criterion, a version identifier, a source identifier, or other information useful for managing candidate evaluation or iterative refinement.

In one embodiment, the system (100) includes an input validation module (130). The input validation module (130) may be configured to receive the DNARP program definition (120), verify that required fields are present, normalize sequence and parameter formats, confirm that the candidate is machine-readable, and reject or flag malformed candidate programs prior to substantive evaluation. For example, the input validation module (130) may verify that the sequence component contains recognizable nucleotides, codons, or grouped sequence blocks, that the shape component contains values corresponding to helical pitch and groove ratio, and that the energy component contains or implies an allowed coherence-state selection and associated rate information. The input validation module (130) may additionally perform syntax checking, field typing, character-set validation, range sanity checking, conversion of alternative sequence-input formats, and tuple normalization operations. In one implementation, the tuple parsing step described in connection with the DNARP evaluation method (300) is at least partially supported by the input validation module (130), such that candidate definition and tuple normalization are handled before more computationally intensive analyses are performed.

In one embodiment, the system (100) further includes a stability analysis module (140). The stability analysis module (140) may be configured to perform one or more of the sequence stability computation, shape-related admissibility processing, and structural stability evaluation operations described elsewhere in the present disclosure. In some implementations, the stability analysis module (140) may include or cooperate with a sequence-scoring engine, a geometry-validation engine, and a structural solver capable of processing the DNA Lagrangian and associated stability criteria. For example, the stability analysis module (140) may compute the sequence stability score from sequence content, verify that the helical pitch and groove ratio lie within disclosed admissibility ranges, instantiate the structural stability formulation using the candidate pitch and relevant implementation parameters, solve or approximate the resulting profile C(r), and determine whether the candidate satisfies the disclosed structural tolerance condition. In other implementations, the stability analysis module (140) may be divided internally into additional submodules while still functioning overall as the stability-processing portion of the system architecture.

In one embodiment, the system (100) includes a coherence validation module (150). The coherence validation module (150) may be configured to determine whether the coherence-related state requested by the candidate DNARP program is valid and admissible under the disclosed DNA recognition operator framework. In one implementation, the coherence validation module (150) receives the candidate coherence level, derives or verifies the associated discrete coherence energy level, determines the normalized coordinate form, instantiates the DNA recognition operator, evaluates the requested state against an allowed coherence state set, and, where appropriate, determines whether a corresponding state function is admissible. The coherence validation module (150) may therefore include or cooperate with one or more normalization routines, operator-evaluation engines, eigenvalue-checking routines, state-function solvers, and admissibility-testing routines. Where the candidate fails coherence validation, the coherence validation module (150) may cause the system to reject the candidate prior to output computation. Where the candidate passes coherence validation, the module may provide a validated coherence state and associated parameters to later stages of the system.

In one embodiment, the system (100) includes a function prediction module (160). The function prediction module (160) may be configured to compute the predicted functional output associated with a candidate DNA program after the candidate has passed the earlier required validation stages. In one implementation, the function prediction module (160) receives the validated sequence stability score, the validated coherence-related state, the associated rate, and the candidate helical pitch, then applies the disclosed recognition transform to determine an output amplitude and final predicted functional output. The function prediction module (160) may include or cooperate with a transform-evaluation engine, a numerical computation routine, a complex-phase handling routine, and one or more output-value generation routines. In some implementations, the function prediction module (160) may additionally compute comparative quantities such as baseline-relative enhancement, target satisfaction status, or ranking values for multiple candidates, provided that the core predicted output remains grounded in the disclosed transform-based formulation.

In one embodiment, the system (100) includes an output module (170). The output module (170) may be configured to provide one or more resulting outputs after candidate evaluation, including an acceptance status, rejection status, predicted functional output, enhancement metric, failure classification, compatibility measure, candidate ranking, or other system-generated result. In one implementation, the output module (170) formats the candidate result for display to a user, transmission through an API, storage in memory or a database, forwarding to a downstream design or laboratory workflow, or return to an optimization engine for additional iterations. The output module (170) may therefore function as both a reporting stage and an interface stage between the disclosed DNARP evaluation framework and other software or laboratory environments. In some embodiments, the output module (170) may also generate logs, audit trails, or candidate histories associated with evaluated DNARP programs.

The module-level architecture shown in (FIG. 1) may be implemented on a variety of computing platforms, one example of which is shown in the computing environment (1200) of (FIG. 12). In one embodiment, the computing environment (1200) includes one or more processors (1210), memory (1220), non-transitory storage (1230), an input interface (1240), an output interface (1250), a network interface (1260), DNARP evaluation instructions (1270), a candidate database (1280), and a remote or cloud execution node (1290). These components collectively provide one suitable hardware and software environment for executing the disclosed DNARP framework, although the invention is not limited to the precise architectural arrangement illustrated in (FIG. 12).

The processor (1210) may include one or more general-purpose processors, application-specific processors, virtual processors, distributed processors, or other computing resources configured to execute the DNARP evaluation instructions (1270). In one embodiment, the processor (1210) executes instructions that cause the system to receive a candidate DNA program, compute one or more sequence-, shape-, and coherence-related metrics, evaluate candidate admissibility under the disclosed constraints, and generate one or more predicted functional outputs or related results. In some implementations, different processors or processing threads may be assigned to different stages of the evaluation pipeline, such as sequence scoring, structural stability solving, coherence validation, and output prediction. In other implementations, the disclosed evaluation flow may be executed serially on a single processor resource. The present disclosure is not limited to any one processor topology so long as one or more processors are configured to perform the disclosed DNARP operations.

The memory (1220) may include volatile memory, non-volatile memory, cache memory, working memory, or other memory resources suitable for storing executable instructions, intermediate values, parsed candidate structures, computed stability profiles, eigenvalue results, output values, or other data used during execution of the DNARP framework. In one implementation, the memory (1220) stores active instances of candidate DNARP program tuples, intermediate sequence counts, normalized geometric values, solver parameters, transform values, and candidate evaluation status information while the system processes a candidate program. The non-transitory storage (1230) may include persistent local or remote storage media suitable for storing executable code, candidate libraries, parameter sets, evaluation histories, accepted candidates, rejected candidates, reference data, or other persistent information associated with operation of the disclosed system. In one embodiment, the DNARP evaluation instructions (1270) are stored in the non-transitory storage (1230) and loaded into memory (1220) for execution by the processor (1210).

The input interface (1240) may include one or more interfaces through which candidate DNA programs, parameter selections, target outputs, or optimization requests are supplied to the system. In one embodiment, the input interface (1240) supports interactive manual entry of sequence and parameter values. In another embodiment, the input interface (1240) supports batch import of candidate files, integration with external design tools, or receipt of API-submitted candidate DNARP programs. The output interface (1250) may include one or more interfaces through which the system provides predicted outputs, validity determinations, candidate rankings, or other results to a user, another software process, or an external computing environment. In one embodiment, the output interface (1250) supports graphical display of candidate evaluation results. In another embodiment, the output interface (1250) supports structured machine-readable output, such as formatted data returned to a calling service or written to persistent storage. Thus, the disclosed system may operate both as an interactive design environment and as a back-end computational engine depending on the selected implementation.

The network interface (1260) may enable the system to communicate with external computing resources, remote data sources, cloud-based execution environments, candidate repositories, laboratory information systems, or other network-connected services. In one embodiment, the disclosed DNARP framework is implemented as a cloud service or distributed computing service in which candidate evaluation requests are received over a network, processed on one or more remote computing nodes, and returned to a client system. In another embodiment, the network interface (1260) is used to retrieve parameter sets, submit evaluation jobs, synchronize candidate libraries, or integrate the disclosed DNARP framework with an external synthetic-biology workflow. Thus, the system architecture is not limited to a standalone local machine and may instead operate in a distributed or hybrid computing environment.

The DNARP evaluation instructions (1270) may include executable instructions which, when executed by the processor (1210), cause the system to implement the disclosed DNARP evaluation method and related embodiments. In one embodiment, these instructions cause the system to receive a DNARP program, parse the program into sequence, shape, and energy components, compute the sequence stability score, validate the shape parameters, evaluate structural stability using the disclosed DNA Lagrangian, validate coherence using the disclosed DNA recognition operator, compute predicted functional output using the disclosed recognition transform, and provide a resulting candidate output. In some embodiments, the DNARP evaluation instructions (1270) may also include instructions for candidate normalization, parameter derivation, candidate-library management, iterative optimization, result ranking, or application-specific workflow handling, such as therapeutic design evaluation, regulatory tuning evaluation, or DNA computing evaluation. These additional functions may be integrated into one software package or may be distributed across cooperating software components without departing from the disclosed architecture.

The candidate database (1280) may be used to store one or more candidate DNARP programs, validated tuples, failed candidates, failure reasons, predicted outputs, target criteria, optimization histories, parameter selections, or other information associated with design exploration and system operation. In one implementation, the candidate database (1280) stores candidate tuples in structured form so that the system may retrieve, compare, re-evaluate, rank, or iteratively refine previously considered candidates. In another implementation, the candidate database (1280) stores both accepted and rejected candidates together with metadata indicating which disclosed validation stage each candidate passed or failed. This may be useful, for example, in large-scale candidate screening, optimization workflows, or audit-trace scenarios in which the system maintains a structured record of how and why a candidate was accepted, rejected, or modified. The present disclosure does not require a particular database technology so long as the system is capable of storing and retrieving candidate-related data in support of the disclosed framework.

The remote or cloud execution node (1290) may represent one or more remote computing resources used to execute some or all of the disclosed DNARP operations. In one embodiment, local preprocessing of candidate input occurs on a client machine, while structural stability solving, coherence validation, or transform-based output computation occurs remotely on a cloud platform or other distributed computing resource. In another embodiment, the remote or cloud execution node (1290) hosts the complete DNARP evaluation service and receives candidate requests over a network through the network interface (1260). In still another embodiment, multiple remote nodes may cooperatively evaluate batches of candidate DNA programs in parallel. This support for remote or distributed execution is useful because the disclosed framework may include solver-based and iterative computations that benefit from scalable computing resources, particularly in large design-space exploration scenarios.

In some embodiments, the disclosed system may additionally include an optimization engine, a syntax-checking engine, a sequence-scoring submodule, a shape-validation submodule, an energy-consistency checking submodule, a ranking module, or a reporting module, whether or not such subcomponents are separately illustrated in the drawings. Such subcomponents may be implemented as discrete software modules, callable services, libraries, processes, threads, containers, virtualized services, or other executable units. The present disclosure does not require that every logical function be implemented as a separately labeled box in the architecture. Rather, the disclosed figures illustrate one suitable architectural arrangement showing that the invention may be implemented in software and hardware, and that the disclosed candidate evaluation logic may be embodied in one or more processors executing stored instructions within a suitable computing environment.

The disclosed system architecture may be implemented as a processor-based system, a storage-medium-based software implementation, or a network-accessible software service. A non-transitory storage medium may store instructions that, when executed, cause one or more processors to perform the disclosed candidate-receipt, tuple-parsing, sequence-analysis, shape-validation, structural-stability, coherence-validation, output-prediction, and result-reporting operations. Likewise, a network-accessible implementation may provide the same functionality as a software service. By describing the DNARP design and simulation system (100) in conjunction with the computing environment (1200), the present disclosure expressly supports implementations in local tools, workstation software, server-based systems, distributed platforms, and cloud services while preserving the same underlying tuple-driven DNA design and evaluation logic.

Accordingly, the system architecture and computing support described herein provide a concrete implementation framework for carrying out the disclosed DNARP invention in software and hardware. By defining the role of the user or API input (110), the DNARP program definition (120), the input validation module (130), the stability analysis module (140), the coherence validation module (150), the function prediction module (160), and the output module (170) within the system (100), and by further defining the processor (1210), memory (1220), non-transitory storage (1230), input interface (1240), output interface (1250), network interface (1260), DNARP evaluation instructions (1270), candidate database (1280), and remote or cloud execution node (1290) within the computing environment (1200), the present disclosure demonstrates that the disclosed DNA programming language, validation framework, and output-prediction method are fully implementable on practical computing systems. This computing support therefore forms an integral part of the invention and provides explicit structural and software support for implementations of the disclosed DNARP framework.

In one embodiment, the disclosed DNARP framework is applied as a therapeutic design workflow (900) for evaluating or designing a candidate therapeutic DNA construct (920) directed to a selected therapeutic target (910). In the present disclosure, this embodiment is workflow-oriented and uses the previously described DNARP language, validation logic, and output-prediction framework as the basis for therapeutic candidate selection. The therapeutic design workflow (900) therefore does not require a separate evaluation theory distinct from the core DNARP framework. Rather, it applies the same tuple-based representation, admissibility constraints, structural stability evaluation, coherence validation, and output-prediction operations to a therapeutic design context in which the design objective is to identify a candidate therapeutic DNA construct (920) predicted to provide a desired level of therapeutic function, therapeutic expression, or target-associated activity.

In one implementation, the workflow begins by identifying the therapeutic target (910). The therapeutic target (910) may be a gene, pathway, expression objective, regulatory objective, disease-associated molecular function, longevity-associated target, metabolic target, cellular repair target, or another therapeutic objective for which a DNA-based construct is to be designed or evaluated. In some embodiments, the therapeutic target (910) may correspond to a target gene whose increased, decreased, or otherwise modulated activity is desired. In some embodiments, the therapeutic target (910) may correspond to a therapeutic payload or a therapeutic expression profile required to achieve a selected dose, response window, or biological effect. In some embodiments, the therapeutic target (910) may correspond to a longevity-associated or precision-medicine-associated objective, such as modulation of a target gene involved in cellular maintenance, mitochondrial function, oxidative stress control, DNA repair, or age-associated metabolic regulation. The disclosed workflow does not require that the therapeutic target (910) be limited to one disease class or one specific therapeutic mechanism, provided that a candidate therapeutic DNA construct (920) can be represented and evaluated under the disclosed DNARP framework.

After the therapeutic target (910) is identified, the workflow defines or selects a candidate therapeutic DNA construct (920) for evaluation. In one implementation, the candidate therapeutic DNA construct (920) is represented as a DNARP program tuple D=(S, H, E), in which the sequence component specifies the ordered sequence content of the therapeutic construct, the shape component specifies the geometric values associated with the candidate construct, and the energy component specifies the requested coherence-related state and associated output rate. The candidate therapeutic DNA construct (920) may correspond to a coding-region construct, a regulatory construct, a hybrid regulatory-plus-coding construct, a therapeutic expression cassette, a payload-supporting construct, or another DNA-based design intended for therapeutic use. In some embodiments, the candidate therapeutic DNA construct (920) may be entered by a designer. In some embodiments, it may be selected from a candidate library. In some embodiments, it may be generated automatically by a design engine, optimization engine, or iterative candidate-generation routine. Regardless of how the candidate therapeutic DNA construct (920) is obtained, the disclosed workflow treats it as a candidate DNARP program that is subjected to the same objective validation-and-prediction stages described elsewhere in the present disclosure.

In one embodiment, the candidate therapeutic DNA construct (920) is first subjected to sequence-level evaluation. The system determines the sequence stability score associated with the candidate construct and assesses whether the sequence component provides sufficient support for continued therapeutic evaluation. In one implementation, the system computes S_stab from the sequence content of the candidate therapeutic DNA construct (920) according to the disclosed weighted contribution model and determines whether the resulting value satisfies the required threshold. If the candidate fails the sequence-level admissibility requirement, the workflow may reject the candidate therapeutic DNA construct (920) before more detailed therapeutic analysis is undertaken. This is useful because a therapeutic construct that lacks sufficient sequence stability under the disclosed framework is not advanced merely because it corresponds to a desirable target. Instead, the disclosed workflow requires that the therapeutic candidate first satisfy the same sequence-validity logic that governs DNARP programs generally.

If the candidate therapeutic DNA construct (920) satisfies the sequence-level requirement, the workflow then evaluates the geometric configuration associated with the construct. In one implementation, the system determines whether the candidate helical pitch and groove ratio associated with the construct fall within the disclosed admissibility ranges. A therapeutic construct directed to the therapeutic target (910) is therefore not treated as geometrically valid merely because it is functionally motivated. Rather, the disclosed workflow requires that the construct also satisfy the shape constraints of the DNARP framework. If the candidate geometry is out of range, the system may reject or flag the candidate therapeutic DNA construct (920) as non-admissible. In some embodiments, geometric closeness to preferred values may also be used as a therapeutic quality or compatibility indicator, especially where the therapeutic design objective includes elevated output or elevated coherence-state selection.

In one embodiment, the workflow next evaluates the energy component of the candidate therapeutic DNA construct (920) by determining whether the requested coherence-related state is consistent with the allowed state logic of the disclosed framework. In one implementation, the candidate therapeutic DNA construct (920) is associated with a selected coherence index n, and the system computes or verifies the corresponding values of E_n and R. A candidate therapeutic DNA construct (920) that requests a coherence-related state outside the allowed discrete set, or that requests a rate inconsistent with the selected state, may be rejected as failing the therapeutic DNARP configuration requirements. This stage is useful because it ensures that the therapeutic workflow does not merely pursue a desired therapeutic outcome in the abstract, but instead grounds the candidate therapeutic DNA construct (920) in a valid coherence-state selection supported by the disclosed tuple framework.

After the candidate therapeutic DNA construct (920) has passed the basic sequence, shape, and state-consistency checks, the workflow applies the previously described structural stability evaluation. In one implementation, the system instantiates the DNA Lagrangian using the candidate pitch and computes or approximates the corresponding stability profile for the candidate therapeutic DNA construct (920). The system then determines whether the computed profile remains within the disclosed tolerance band. If the candidate fails this structural stability criterion, the candidate therapeutic DNA construct (920) may be rejected as insufficiently stable for further therapeutic evaluation under the disclosed framework. In contrast, if the candidate satisfies the structural stability criterion, the candidate is permitted to proceed to coherence validation. This stage is particularly useful in the therapeutic context because it ensures that a candidate therapeutic DNA construct (920) is not advanced based solely on target relevance or sequence-level plausibility, but is also supported by a structurally admissible helix-associated profile.

In one embodiment, the workflow then performs coherence validation for the candidate therapeutic DNA construct (920). At this stage, the system determines whether the requested coherence-related state is an admissible operator-governed state for the therapeutic candidate configuration. In one implementation, the system applies the DNA recognition operator to the candidate state, determines whether the requested discrete coherence level belongs to the allowed set, and optionally determines whether a corresponding state function is admissible. If the candidate therapeutic DNA construct (920) fails coherence validation, the workflow rejects or flags the candidate as non-admissible notwithstanding earlier successful sequence, shape, and structural checks. If the candidate passes coherence validation, the candidate is treated as therapeutically coherence-valid and is eligible for final output prediction. This stage is useful because it grounds the therapeutic design workflow (900) in the same coherence-state logic used throughout the disclosed DNARP framework, thereby preserving a unified technical basis for therapeutic candidate evaluation.

Once the candidate therapeutic DNA construct (920) has passed the required validation stages, the workflow computes a predicted functional output associated with the candidate construct. In one implementation, the system applies the disclosed recognition transform using the validated sequence stability score, validated coherence level, associated rate, and candidate helical pitch to determine a predicted output value for the therapeutic construct. This predicted output may represent a predicted therapeutic expression-related output, a predicted construct-associated functional output, a baseline-relative enhancement, or another predicted measure relevant to the therapeutic design objective. The disclosed workflow therefore provides a deterministic basis for deciding whether the candidate therapeutic DNA construct (920) is likely to satisfy a selected therapeutic design criterion under the disclosed framework. In some embodiments, the output may be compared to a target therapeutic threshold, target dose-related output criterion, or target enhancement criterion associated with the therapeutic target (910).

In one implementation, the therapeutic design workflow (900) may use the predicted output to classify the candidate therapeutic DNA construct (920) as accepted, rejected, or suitable for refinement. If the candidate satisfies the required admissibility conditions and its predicted output meets or exceeds the relevant therapeutic objective, the system may accept the candidate therapeutic DNA construct (920) as a therapeutically suitable construct under the disclosed DNARP framework. If the candidate satisfies the admissibility conditions but fails to meet the therapeutic output objective, the workflow may classify the construct as valid but sub-target, thereby indicating that the construct remains a valid DNARP candidate yet should be refined or replaced to better satisfy the therapeutic target (910). If the candidate fails any required validity condition, the workflow may reject the candidate and optionally identify the failed stage, such as sequence insufficiency, geometric non-admissibility, structural instability, or coherence invalidity.

In one embodiment, the therapeutic design workflow (900) may include an iterative refinement stage in which a rejected or sub-target candidate therapeutic DNA construct (920) is modified and re-evaluated. For example, if the candidate fails the sequence threshold, the system may alter one or more codons or sequence units to improve S_stab. If the candidate is sequence-valid but output-deficient, the system may alter sequence content, target a different permitted coherence state, or select a more conforming geometric configuration. If the candidate is structurally valid but coherence-invalid, the system may adjust the candidate to improve compatibility between the sequence, geometry, and requested state. After any such modification, the revised candidate therapeutic DNA construct (920) may be reintroduced into the therapeutic design workflow (900) and reevaluated using the same disclosed DNARP stages. This iterative use is useful because therapeutic design often requires selection among multiple candidate constructs rather than evaluation of a single fixed construct.

In one therapeutic use case, the therapeutic target (910) corresponds to a longevity-associated gene or pathway, and the candidate therapeutic DNA construct (920) is designed to provide elevated output at an allowed coherence state consistent with the disclosed framework. In such an embodiment, the system may evaluate whether a candidate construct designed to increase target-associated output at n=2 or n=3 possesses sufficient sequence stability, acceptable geometry, structural admissibility, and coherence validity to justify the requested enhanced state.

A construct that lacks sufficient sequence support or geometric conformity for the higher requested state may be rejected, whereas a construct satisfying the disclosed thresholds and operator-based criteria may be accepted and assigned a predicted enhancement level. This use case is consistent with the disclosed therapeutic and longevity-oriented applications of the DNARP framework while keeping the workflow centered on the previously described tuple-and-simulator logic.

In another therapeutic use case, the therapeutic target (910) corresponds to a therapeutic expression objective requiring a selected output level associated with dosing or efficacy. In such an embodiment, the candidate therapeutic DNA construct (920) may be evaluated not merely for binary validity but for whether its predicted functional output is sufficient for the intended therapeutic design objective. A candidate construct having an otherwise valid DNARP configuration may be deemed therapeutically insufficient if its predicted output remains below the selected objective, while another construct with stronger sequence support or a different allowed coherence state may satisfy the therapeutic requirement. The therapeutic design workflow (900) is therefore capable of functioning both as a validity filter and as a design-selection workflow for therapeutically relevant candidate constructs.

The therapeutic design workflow (900) shown in (FIG. 9) thus provides a lean, application-specific embodiment of the broader DNARP framework. The therapeutic target (910) represents the selected therapeutic objective to be addressed, and the candidate therapeutic DNA construct (920) represents the DNARP-defined construct being evaluated against that objective. Although (FIG. 9) is intentionally workflow-oriented rather than heavily anatomical, it still supports a complete therapeutic design process in which a therapeutic target is identified, a candidate construct is defined, the candidate is evaluated using the disclosed DNARP validation-and-prediction framework, and the candidate is accepted, rejected, or refined based on the resulting determinations. This preserves alignment between the therapeutic application embodiment and the core invention, which remains a formal DNA programming language, simulation system, and computer-implemented evaluation method rather than a patent limited to one specific therapeutic construct architecture.

Accordingly, the therapeutic design workflow embodiment described herein demonstrates how the disclosed DNARP framework may be used in a therapeutic context without departing from the common tuple-driven and simulator-driven logic of the invention. By identifying a therapeutic target (910), defining a candidate therapeutic DNA construct (920), evaluating that construct using the disclosed sequence, shape, structural stability, coherence, and output stages, and then accepting, rejecting, or refining the construct based on the resulting validity and predicted-output determinations, the workflow (900) provides a concrete and machine-implementable therapeutic application of the disclosed DNA programming framework.

In one embodiment, the disclosed DNARP framework is applied as a regulatory element tuning workflow (1000) for designing, evaluating, selecting, and iteratively refining a candidate promoter or regulatory element (1010) toward a selected target output criterion (1030). In the present disclosure, this embodiment is workflow-oriented and uses the same DNARP tuple representation, validation logic, structural stability evaluation, coherence validation, and transform-based output prediction previously described for the core framework. The regulatory element tuning workflow (1000) therefore does not introduce a separate design theory for promoters or regulatory elements. Rather, it applies the common DNARP formalism to a design context in which the principal objective is to identify, from among one or more sequence variants, a candidate promoter or regulatory element (1010) predicted to satisfy a selected functional objective while remaining admissible under the disclosed sequence, geometry, and coherence constraints.

In one implementation, the workflow begins by defining the candidate promoter or regulatory element (1010) that is to be tuned. The candidate promoter or regulatory element (1010) may correspond to a promoter region, enhancer-associated region, operator-containing sequence, transcription-control region, synthetic regulatory cassette, hybrid control region, or another DNA sequence element whose activity is to be evaluated or adjusted. In some embodiments, the candidate promoter or regulatory element (1010) is provided as an initial starting design entered by a user. In some embodiments, it is selected from a stored set of known or proposed control-region sequences. In some embodiments, it is generated by a design engine or optimization routine. Regardless of origin, the candidate promoter or regulatory element (1010) is treated as a candidate DNARP program or as the sequence-bearing portion of a candidate DNARP program capable of being expressed in the tuple form D=(S, H, E) and subjected to the disclosed evaluation framework.

After the candidate promoter or regulatory element (1010) is defined, the workflow may generate, receive, or identify a sequence variant set (1020). The sequence variant set (1020) may include a plurality of alternative regulatory sequences differing by one or more substitutions, insertions, deletions, motif rearrangements, codon-level changes where applicable, block-level substitutions, or other sequence modifications intended to alter regulatory behavior under the disclosed framework. In one embodiment, the sequence variant set (1020) is produced by systematically modifying one or more subsequences of the candidate promoter or regulatory element (1010). In another embodiment, the sequence variant set (1020) is selected from a candidate library containing multiple promoter or regulatory-element variants. In another embodiment, the sequence variant set (1020) is generated iteratively during operation of the workflow, such that each accepted, rejected, or sub-target result informs the selection of a next candidate variant. The sequence variant set (1020) therefore provides the design-space basis for regulatory tuning, allowing the system to compare multiple candidate regulatory sequences under a common DNARP evaluation framework.

The workflow also defines a target output criterion (1030) against which the candidate promoter or regulatory element (1010) and the sequence variant set (1020) are evaluated. The target output criterion (1030) may correspond to a desired promoter strength, a desired transcription-related output level, a desired regulatory activation level, a desired repression level, a desired baseline-relative enhancement, or another selected functional measure expressible through the disclosed output-prediction framework. In one embodiment, the target output criterion (1030) is a minimum acceptable predicted functional output. In one embodiment, the target output criterion (1030) is a range-bound target within which the candidate is desired to fall. In one embodiment, the target output criterion (1030) is defined relative to a baseline promoter or baseline regulatory construct, such that the workflow seeks a candidate predicted to provide a specified fold enhancement or fold reduction relative to the baseline. In another embodiment, the target output criterion (1030) may be associated with a coherence-state selection, such that the workflow seeks a candidate capable of valid operation at a specified allowed coherence level while also satisfying a minimum output objective. In each case, the target output criterion (1030) provides an objective design goal against which the disclosed workflow may accept, reject, or refine candidate regulatory sequences.

In one embodiment, each candidate in the sequence variant set (1020) is converted into or associated with a DNARP tuple representation and is evaluated beginning with the sequence component. The system computes a sequence stability score for the candidate promoter or regulatory element (1010) or for a selected variant in the sequence variant set (1020) according to the disclosed weighted sequence-stability formulation. In one implementation, the system counts CG-supported and AT-supported contributions within the candidate regulatory sequence and computes S_stab using the same relation previously described for the sequence component.

The resulting value is compared against the disclosed sequence threshold. If the candidate variant fails the threshold, the workflow may reject that variant from further consideration. This is useful because a promoter or regulatory element is not treated as tunable solely on the basis of desired activity. Rather, the disclosed workflow requires that the candidate also possess adequate sequence-level support under the DNARP framework before further structural and coherence-based evaluation proceeds.

If the candidate promoter or regulatory element (1010) or a selected sequence variant satisfies the sequence threshold, the workflow then evaluates the associated shape component. In one implementation, the system determines whether the candidate helical pitch and groove ratio lie within the disclosed admissibility ranges and, where appropriate, how closely the candidate geometry conforms to preferred values such as P_0 and G_0. Because regulatory behavior is evaluated within the same formal DNARP framework used for other candidate DNA designs, geometric admissibility is treated as a meaningful gating condition rather than as a purely optional design annotation. A candidate regulatory variant having sequence content sufficient to pass the sequence threshold may still be rejected if its associated geometry is outside the disclosed pitch or groove-ratio range. In some embodiments, closeness to the preferred geometry may also be used as a tuning preference, such that among multiple otherwise valid variants, those with better geometric conformity are favored for higher coherence-level requests or stronger predicted output objectives.

In one embodiment, the workflow further evaluates the energy component associated with each candidate regulatory variant. In one implementation, the system determines whether the requested or assigned coherence-related state is within the allowed discrete set and whether the associated rate is consistent with the selected coherence level. Thus, the workflow may evaluate whether a candidate promoter or regulatory element (1010) is intended to operate at n=1, n=2, or n=3, and may reject a candidate that requests a state outside the allowed set or an inconsistent rate. This stage is useful because the regulatory tuning workflow (1000) is not limited to sequence exploration alone. Rather, it permits exploration of whether a regulatory candidate is compatible with a desired discrete coherence-related operating regime under the same tuple-based logic used elsewhere in the disclosed invention.

After the candidate has passed the sequence, geometry, and basic energy-consistency stages, the workflow performs structural stability evaluation. In one implementation, the system applies the disclosed DNA Lagrangian using the candidate helical pitch and relevant implementation parameters, computes or approximates the corresponding stability profile, and determines whether the profile remains within the disclosed stability tolerance band. A regulatory variant that fails the structural stability criterion is rejected from further tuning consideration, even if it nominally satisfies the earlier sequence and geometry ranges. A regulatory variant that passes structural stability may proceed to coherence validation. This stage is useful because it ensures that candidate regulatory designs are not selected merely for achieving a predicted output target in the abstract, but instead are selected from among candidates that remain structurally admissible under the disclosed governing construct.

In one embodiment, the workflow next performs coherence validation for the candidate promoter or regulatory element (1010). At this stage, the system applies the disclosed DNA recognition operator to determine whether the candidate-requested coherence state is an admissible state for the regulatory candidate configuration. In one implementation, the system determines whether the selected discrete coherence level belongs to the allowed coherence state set and whether a corresponding state function is admissible under the disclosed operator framework. A regulatory variant that fails coherence validation is removed from further consideration or is flagged as unsuitable for the selected operating regime. A regulatory variant that passes coherence validation is eligible for final output prediction. This coherence-validation stage is important in the tuning workflow because it prevents the selection of regulatory candidates whose requested output regime is unsupported by the disclosed operator-governed state logic.

Once a candidate promoter or regulatory element (1010) has passed the required admissibility stages, the workflow computes a predicted functional output for that candidate using the disclosed recognition transform. In one implementation, the system applies the transform-based output formulation to the validated candidate state to determine a predicted output value associated with the candidate regulatory sequence. The predicted output may correspond to a predicted promoter-associated output, a predicted regulatory activity level, a predicted activation strength, a predicted repression-related output measure, or another regulatory-effect quantity consistent with the disclosed framework. The predicted value is then compared against the target output criterion (1030). If the predicted output satisfies the target output criterion (1030), the candidate may be retained as a successful tuning result. If the predicted output does not satisfy the target output criterion (1030), the candidate may be classified as valid but sub-target, thereby indicating that the candidate remains admissible under the DNARP framework but does not yet satisfy the selected regulatory design objective.

The iterative tuning loop (1070) provides the mechanism by which sub-target or otherwise improvable candidates may be refined. In one embodiment, the iterative tuning loop (1070) operates by selecting one or more candidates from the sequence variant set (1020), modifying one or more sequence units, and reevaluating the modified candidate under the same DNARP workflow. For example, the system may alter local sequence content to increase the sequence stability score, improve compatibility with a selected coherence level, or more closely align the candidate with the target output criterion (1030). In another embodiment, the iterative tuning loop (1070) may preserve the sequence and instead evaluate a different allowed coherence level for the same candidate. In another embodiment, the iterative tuning loop (1070) may preserve the sequence and coherence target while adjusting or selecting a more suitable geometric assignment within the disclosed admissibility limits. In still another embodiment, the iterative tuning loop (1070) may rank multiple candidates and then apply additional modifications only to the highest-performing variants. The iterative tuning loop (1070) therefore allows the workflow to function as an optimization process rather than only as a single-pass validator.

In one implementation, the workflow may use explicit decision rules within the iterative tuning loop (1070). A candidate regulatory variant may be rejected if it fails the sequence threshold, rejected if it falls outside the disclosed pitch range, rejected if it falls outside the disclosed groove-ratio range, rejected if it requests an invalid coherence level, rejected if it fails structural stability, or rejected if it fails coherence validation. A candidate that passes those validity checks but fails to meet the target output criterion (1030) may be retained in the loop for further refinement rather than discarded outright. In one embodiment, the workflow may terminate when a candidate satisfies all required admissibility conditions and meets or exceeds the target output criterion (1030). In another embodiment, the workflow may terminate when no additional improvement is achieved, when a maximum number of iterations is reached, when the highest-ranked candidate stabilizes across repeated evaluations, or when another selected convergence rule is satisfied.

The optimized regulatory element (1080) represents a candidate promoter or regulatory sequence selected by the workflow as satisfying the chosen tuning objective under the disclosed framework. In one embodiment, the optimized regulatory element (1080) is the first candidate to pass all required validity checks and satisfy the target output criterion (1030). In one embodiment, the optimized regulatory element (1080) is the highest-ranked candidate among multiple valid candidates that all meet the target output criterion (1030). In one embodiment, the optimized regulatory element (1080) is a candidate that does not necessarily produce the absolute highest predicted output, but instead best matches a constrained target range or best balances output with structural or coherence margin. Thus, the optimized regulatory element (1080) may be selected according to absolute output, target matching, stability reserve, coherence compatibility, or a combination of such considerations, so long as the candidate remains grounded in the disclosed DNARP evaluation framework.

In one use case, the regulatory element tuning workflow (1000) is used to design a promoter sequence capable of achieving a selected elevated output relative to a baseline promoter. The sequence variant set (1020) may include multiple promoter variants differing in motif composition, local sequence substitutions, or grouped sequence-block arrangements. The system evaluates each variant for sequence admissibility, geometric admissibility, structural stability, coherence validity, and predicted output. Variants failing any required validity stage are removed.

Variants that pass but remain below the target output criterion (1030) are iteratively modified through the tuning loop (1070). The optimized regulatory element (1080) is then selected as the variant predicted to satisfy the target output objective while remaining valid under the disclosed sequence-shape-energy framework. This use case is consistent with promoter tuning, synthetic regulatory design, and broader regulatory element engineering under the DNARP system.

In another use case, the regulatory element tuning workflow (1000) is used not to maximize output, but to tune a regulatory element toward a bounded target window. For example, a user may seek a promoter or regulatory element whose predicted output falls within a specified range rather than exceeding a maximum threshold. In such an embodiment, the target output criterion (1030) may be a bounded interval, and the iterative tuning loop (1070) may seek candidates whose transform-based predicted output converges toward that interval. A candidate producing excessive output may therefore be treated as sub-optimal even if it is fully valid, while another candidate with a more moderate predicted output may be selected as the optimized regulatory element (1080). This illustrates that the workflow supports not only enhancement-driven optimization, but also target-matching optimization under the same disclosed framework.

In another use case, the workflow may be applied to a regulatory element associated with a therapeutic or cellular-control context, provided that the candidate remains represented and evaluated under the same tuple-based DNARP formalism. For example, a regulatory region associated with therapeutic gene control may be tuned so that its predicted output satisfies a therapeutic objective while also remaining valid under the disclosed sequence, geometry, stability, and coherence constraints. In such an embodiment, the regulatory element tuning workflow (1000) may operate in conjunction with, or as a precursor to, the therapeutic design workflow previously described, while still maintaining a distinct focus on tuning the regulatory sequence itself. This further illustrates that the application workflows disclosed herein are related embodiments of the same DNARP framework rather than isolated inventions.

The regulatory element tuning workflow (1000) shown in (FIG. 10) therefore provides a lean but technically complete application-specific embodiment of the disclosed invention. The candidate promoter or regulatory element (1010) provides the starting design object, the sequence variant set (1020) provides the candidate exploration space, the target output criterion (1030) provides the objective for tuning, the iterative tuning loop (1070) provides the refinement mechanism, and the optimized regulatory element (1080) provides the selected design result. Although (FIG. 10) is intentionally streamlined and does not depict every internal metric or intermediate variable, it supports a complete regulatory design process in which candidates are represented in the DNARP language, evaluated using the disclosed simulator framework, refined through iterative design, and selected according to objective validity and predicted-output criteria.

Accordingly, the regulatory element tuning workflow embodiment described herein demonstrates how the disclosed DNARP framework may be used to design and optimize promoters and related regulatory DNA elements without departing from the common tuple-driven and simulator-driven logic of the invention. By defining a candidate promoter or regulatory element (1010), evaluating one or more candidates from a sequence variant set (1020), comparing predicted outputs to a target output criterion (1030), iteratively refining candidates through the tuning loop (1070), and selecting an optimized regulatory element (1080), the workflow (1000) provides a concrete and machine-implementable regulatory design application of the disclosed DNA programming framework. This embodiment therefore supports promoter engineering, regulatory-element tuning, and related synthetic-biology uses of the invention while remaining tightly aligned with the core DNARP validation-and-prediction architecture.

In one embodiment, the disclosed DNARP framework is applied as a DNA computing workflow (1100) for evaluating, selecting, and refining a toehold switch or DNA logic construct (1110) in view of a trigger-responsive switching objective. In the present disclosure, this embodiment is workflow-oriented and applies the same tuple-based representation, admissibility logic, structural stability evaluation, coherence validation, and transform-based output prediction previously described for the core DNARP framework. The DNA computing workflow (1100) therefore does not rely on a separate or unrelated theory of molecular logic. Rather, it treats a toehold switch or DNA logic construct (1110) as a candidate DNARP program, or as a construct capable of being represented by a candidate DNARP program, and evaluates whether that construct exhibits admissible OFF-state and ON-state behavior under the disclosed sequence, shape, and energy framework.

In one implementation, the workflow begins by defining the toehold switch or DNA logic construct (1110). The toehold switch or DNA logic construct (1110) may be a DNA-based or DNA-associated molecular logic element configured to alter its functional state in response to a selected input sequence, a strand-displacement event, a gate-opening event, or another molecular trigger condition. In some embodiments, the construct includes a gate sequence region (1130) that governs whether the construct is in an OFF state (1140) or an ON state (1150). In some embodiments, the construct is configured so that a trigger input sequence (1120) interacts with the gate sequence region (1130) to alter accessibility, activation, or effective output of the construct. In this manner, the disclosed DNA computing workflow (1100) provides a formal way to evaluate molecular switching behavior before physical synthesis or wet-lab implementation.

After the toehold switch or DNA logic construct (1110) is defined, the workflow identifies the trigger input sequence (1120). The trigger input sequence (1120) may be a nucleic acid sequence, a complementary strand segment, an input signal strand, or another sequence-bearing molecular input capable of interacting with the construct. In one embodiment, the trigger input sequence (1120) is selected to bind or otherwise interact with the gate sequence region (1130) so as to change the operational state of the construct from the OFF state (1140) to the ON state (1150). In another embodiment, the trigger input sequence (1120) is selected from among multiple candidate inputs, and the disclosed workflow is used to determine which input sequence is most compatible with the target switching behavior under the DNARP framework. The trigger input sequence (1120) may therefore be treated as part of the candidate design context rather than as an after-the-fact laboratory condition.

The workflow also identifies the gate sequence region (1130) of the toehold switch or DNA logic construct (1110). The gate sequence region (1130) may be the sequence portion responsible for maintaining a repressed or constrained condition in the absence of the trigger input sequence (1120), and for permitting an activated or released condition when appropriately engaged by the trigger input sequence (1120). In one embodiment, the gate sequence region (1130) contributes to the OFF state (1140) by maintaining a lower-output or repressed configuration. In one embodiment, interaction between the trigger input sequence (1120) and the gate sequence region (1130) produces the ON state (1150) by increasing accessibility, increasing stability of an active configuration, or otherwise changing the effective DNARP-evaluated operating condition of the construct. The precise molecular arrangement of the gate sequence region (1130) may vary by embodiment, but within the present disclosure it is sufficient that the gate sequence region (1130) represents the sequence-bearing portion of the construct whose interaction with the trigger input sequence (1120) determines the switching behavior to be evaluated.

In one embodiment, the OFF state (1140) and the ON state (1150) are each treated as candidate DNARP-evaluable states associated with the same overall construct. In one implementation, the OFF state (1140) corresponds to a repressed baseline configuration of the toehold switch or DNA logic construct (1110), while the ON state (1150) corresponds to a triggered configuration generated when the trigger input sequence (1120) appropriately interacts with the gate sequence region (1130). Each such state may be represented as a candidate tuple D=(S, H, E), or may be translated into corresponding sequence, shape, and energy selections for evaluation under the disclosed DNARP framework. Thus, the workflow may compare the OFF state (1140) and ON state (1150) using the same stability, geometry, coherence, and output formulations already described for the broader invention.

In one implementation, the workflow first evaluates the OFF state (1140). The system determines the sequence component associated with the repressed or baseline configuration, computes the corresponding sequence stability score, and determines whether the baseline state is admissible under the disclosed sequence-threshold logic. In one embodiment, the OFF state (1140) is a minimally valid baseline configuration used for comparison against a triggered ON state. For example, an OFF state may be represented as D_off=(S_off, H_off, E_off), where S_off yields #CG pairs=0 and #AT pairs=3 within a predicted paired region such that S_stab_off=1.5*0+1.0*3=3.0, H_off=(P=35, G=1.62), and E_off selects n=1 such that E 1=1*E_coh and R=1*R_0. When the OFF-state configuration satisfies the structural stability criterion (e.g., abs (C(r)−1)<=0.05) and the coherence validation corresponds to an allowed state for n=1, the OFF state is retained as a valid baseline condition. A candidate OFF state (1140) that fails the required sequence threshold or fails stability/coherence validation may be rejected as an invalid baseline state.

If the OFF state (1140) satisfies the required sequence-level admissibility condition, the workflow may then evaluate the associated shape component and energy component for that state. In one implementation, the system determines whether the helical pitch and groove ratio associated with the baseline state fall within the disclosed admissibility ranges, and whether the baseline state is associated with an allowed coherence level and corresponding rate. This is useful because the OFF state (1140) is not treated as an informal null condition outside the scope of the DNARP framework. Rather, it is evaluated as a formally representable and admissible candidate state. A baseline state that is sequence-valid but geometrically non-admissible, coherence-invalid, or structurally unstable may therefore be rejected as an unsuitable OFF-state design for the construct.

The workflow then evaluates the ON state (1150), which corresponds to the triggered or activated configuration of the construct when the trigger input sequence (1120) appropriately engages the gate sequence region (1130). In one implementation, the ON state (1150) is associated with a triggered sequence configuration having greater effective sequence support and a higher predicted output than the OFF state (1140). For example, an ON state may be represented as D_on=(S_on, H_on, E_on), where S_on corresponds to a triggered paired-region configuration having #CG pairs=5 and #AT pairs=0 such that S_stab_on=1.5*5+1.0*0=7.5, H_on=(P=34, G=1.60), and E_on selects n=2 such that E_2=2*E_coh and R=2*R_0.

When the ON-state configuration satisfies the disclosed structural stability criterion (e.g., abs (C(r)−1)<=0.05) and coherence validation corresponds to an allowed state for n=2, the system computes a predicted output for the ON state using the same output formulation used elsewhere in the invention.

In one embodiment, the sequence component of the ON state (1150) is first evaluated to determine whether the triggered configuration satisfies the disclosed sequence threshold and whether its stability score supports the desired switching objective. In the foregoing ON-state example, S_stab_on=7.5 represents a comparatively strong sequence-support value relative to the baseline state. The system may then evaluate the shape component for the ON state (1150), for example by determining whether P=34 and G=1.60 satisfy the disclosed geometric admissibility conditions. The system may further determine whether the selected coherence-related state is valid, such as by confirming that E_2=2*E_coh and R=2*R_0 are consistent with an allowed coherence index n=2. Thus, the ON state (1150) is not merely identified by the existence of a trigger. Rather, it is evaluated as a fully specified and admissible DNARP configuration.

In one implementation, both the OFF state (1140) and the ON state (1150) are further evaluated using the disclosed structural stability and coherence-validation stages. For each state, the system may instantiate the DNA Lagrangian using the candidate pitch, compute or approximate a corresponding stability profile, and determine whether the state remains within the disclosed tolerance band. The system may also instantiate the DNA recognition operator and determine whether the requested coherence state is operator-admissible. This is useful because it ensures that the difference between OFF-state and ON-state behavior is grounded in formally admissible candidate states rather than in uncontrolled or unsupported assumptions. Thus, both states may be required to satisfy the same general structural and coherence requirements, even though they produce different predicted outputs.

Once the OFF state (1140) and ON state (1150) have been validated, the workflow computes the predicted functional output associated with each state using the disclosed recognition transform.

In one implementation, the system computes the output for the ON state according to Output (D)=(S_stab/3)\hat{ }2*n*R_0, using the validated S_stab, n, and R_0 values for the triggered configuration. Using the example above, the triggered-state output may be computed as (7.5/3)\hat{ }2*100=625. The system likewise computes the output for the OFF state (1140). In one directionally disclosed example, the untriggered baseline or repressed state yields Output=1.0*50=50, corresponding to a lower-output baseline condition. These two output values provide a first-principles basis for comparing the dynamic behavior of the toehold switch or DNA logic construct (1110) before synthesis.

In one embodiment, the workflow then determines a switching-performance measure by comparing the ON-state output to the OFF-state output. In one implementation, the switching-performance measure is an ON/OFF ratio defined by dividing the predicted ON-state output by the predicted OFF-state output. Using the directionally disclosed example values above, the resulting ON/OFF ratio is 625/50=12.5x. This is useful because it allows the disclosed workflow to estimate dynamic range, trigger responsiveness, or switching contrast for the construct based on the same tuple-driven and transform-based framework used elsewhere in the invention. The workflow therefore provides a deterministic, machine-generated way to estimate whether a proposed toehold switch or DNA logic construct is likely to exhibit sufficient separation between OFF-state and ON-state operation.

In one implementation, the disclosed DNA computing workflow (1100) may accept, reject, or refine the toehold switch or DNA logic construct (1110) based on the evaluated OFF-state and ON-state behavior. A construct may be accepted if the OFF state (1140) is valid, the ON state (1150) is valid, and the resulting ON/OFF ratio or other switching-performance measure satisfies a selected design objective. A construct may be rejected if either state fails one of the required DNARP admissibility stages, such as sequence insufficiency, geometric non-admissibility, structural instability, or coherence invalidity. A construct may also be classified as valid but sub-target if both states are admissible but the predicted ON/OFF separation remains below a desired threshold. This classification is useful in molecular-logic design because a construct may be formally well-posed yet still need further refinement to achieve sufficient switching contrast.

In one embodiment, the workflow may include an iterative refinement stage in which the toehold switch or DNA logic construct (1110), the trigger input sequence (1120), or the gate sequence region (1130) is modified and reevaluated. For example, if the OFF state (1140) is too high-output, the gate sequence region (1130) may be altered to strengthen repression or reduce baseline stability contributions. If the ON state (1150) is too weak, the trigger input sequence (1120) or the triggered-state sequence content may be altered to improve sequence support, enhance compatibility with an allowed coherence level, or improve predicted output under the disclosed framework. If the ON/OFF ratio is insufficient, one or both states may be modified and reevaluated until an acceptable switching metric is achieved or until a selected iteration limit or termination rule is reached. This iterative use is consistent with the disclosed invention's broader support for design-space exploration and candidate refinement.

In one use case, the DNA computing workflow (1100) is applied to a molecular biosensing or logic-gate context in which a specific trigger input sequence (1120) is intended to activate the toehold switch or DNA logic construct (1110). The system defines the OFF state (1140) in the absence of the trigger and the ON state (1150) in the presence of the trigger, evaluates both states using the disclosed DNARP framework, and computes a predicted switching metric. Constructs predicted to have inadequate ON/OFF separation are rejected or refined, while constructs predicted to have sufficient switching contrast are selected for further development. In another use case, the workflow is applied to a DNA data-processing or strand-displacement context in which the gate sequence region (1130) and trigger input sequence (1120) are designed to produce state-selective behavior under predefined DNARP constraints. These use cases remain within the same formal DNA programming and simulation framework and do not require a separate computational paradigm.

The DNA computing workflow (1100) shown in (FIG. 11) therefore provides a lean but technically complete application-specific embodiment of the disclosed invention. The toehold switch or DNA logic construct (1110) provides the candidate molecular-logic architecture, the trigger input sequence (1120) provides the activating or switching input, the gate sequence region (1130) provides the state-governing region of the construct, and the OFF state (1140) and ON state (1150) provide the two principal operating conditions to be evaluated under the DNARP framework. Although (FIG. 11) is intentionally streamlined and does not depict every intermediate variable or internal solver operation, it supports a complete DNA-computing design process in which molecular-logic candidates are represented, evaluated, compared, and refined using the same tuple-driven and simulator-driven logic as the rest of the disclosed invention.

Accordingly, the DNA computing workflow embodiment described herein demonstrates how the disclosed DNARP framework may be used to evaluate and design toehold switches and related DNA logic constructs without departing from the common tuple-based and validation-based structure of the invention. By defining a toehold switch or DNA logic construct (1110), identifying a trigger input sequence (1120) and a gate sequence region (1130), evaluating OFF-state behavior (1140) and ON-state behavior (1150) under the disclosed sequence, shape, stability, coherence, and output framework, and accepting, rejecting, or refining the construct according to the resulting switching-performance determination, the workflow (1100) provides a concrete and machine-implementable DNA-computing application of the disclosed DNA programming system.

In additional embodiments, the disclosed DNARP framework may be adapted to other nucleic-acid design contexts while preserving the same general tuple-and-simulator architecture described herein. These additional embodiments are not presented as separate inventions with unrelated governing logic. Rather, they are variations in which the same general formal representation, admissibility structure, validation flow, and output-prediction logic are applied to related molecular design problems that are directionally described by the present disclosure.

In one additional embodiment, the disclosed framework may be adapted for RNA design by replacing thymine-containing sequence representations with uracil-containing sequence representations and by using RNA-corresponding parameter values in place of the DNA-specific values described above. In such an embodiment, the sequence component may be represented using RNA sequence units, the shape component may use RNA-applicable geometric values, and the energy component may use coherence-related and rate-related parameters corresponding to an RNA implementation. The same general formal-language logic may therefore be retained, with the candidate program still represented as a sequence-bearing component, a shape-bearing component, and an energy-bearing component evaluated through the disclosed simulator workflow. This RNA-oriented adaptation may be useful for coding RNA, non-coding RNA, guide RNA, regulatory RNA, or other RNA-based design contexts so long as the disclosed tuple-and-validation framework is preserved.

In another additional embodiment, the sequence component may be extended to include epigenetic markup information associated with one or more sequence units. In such an embodiment, the sequence representation may include, in addition to nucleotide or codon identity, one or more epigenetic-state indicators such as methylation state or another chemical-marking state associated with the candidate sequence. The sequence-stability and compatibility computations may then take into account such epigenetic-state information using additional weighting or modification logic consistent with the disclosed framework. In this way, the disclosed DNARP language may be extended beyond bare nucleotide identity while still preserving the core concept that a candidate program is represented in a structured, machine-usable form and evaluated according to objective constraints. Such an embodiment may be useful where the function of the candidate design depends not only on primary sequence content but also on chemically modified sequence state.

In another additional embodiment, the disclosed framework may be extended to multi-strand or composite nucleic-acid designs. In such an embodiment, the sequence-bearing portion of the candidate program may include multiple coordinated sequence components, such as a first strand and a second strand, while the candidate remains evaluable under a common geometry-and-energy framework. By way of example, the disclosed formalism may be used for duplex designs, duplex-plus-overhang designs, strand-displacement architectures, toehold-associated multi-strand constructs, or related multi-part nucleic-acid systems. In one such embodiment, multiple sequence subcomponents may be treated as jointly contributing to a composite candidate representation whose structural stability, coherence admissibility, and predicted output are then evaluated under the disclosed simulator logic. This extension is consistent with the broader DNARP concept that complex genetic or molecular programs may be built from coordinated sequence-bearing design objects rather than only single linear sequence instances.

In another additional embodiment, the disclosed framework may be applied to CRISPR-related guide or regulatory design. In such an embodiment, the sequence component may represent a candidate guide sequence or guide-associated regulatory sequence, the shape component may represent one or more geometry-related constraints associated with the guide-target context, and the energy component may represent a selected coherence-related state and associated rate relevant to the design objective. The disclosed sequence-stability logic may be used to evaluate complementarity-supporting or target-supporting sequence structure, while the disclosed geometry and coherence logic may be used to evaluate whether the candidate guide-related design is admissible under the same general formal framework used for other DNARP programs.

Such an embodiment may be useful for guide-sequence selection, guide-associated regulatory design, or related targeted-editing or targeted-recognition contexts, provided that the candidate remains represented and evaluated under the disclosed tuple-and-simulator architecture.

These additional embodiments are intentionally stated at a general level so that they remain aligned with the presently disclosed invention and do not introduce unsupported new matter. In each case, the adaptation preserves the central technical character of the disclosed DNARP framework: a formal sequence-shape-energy representation, a deterministic validation-and-prediction workflow, and a machine-implementable system for evaluating candidate nucleic-acid designs. Accordingly, RNA embodiments, epigenetic-markup embodiments, multi-strand embodiments, and CRISPR-related embodiments may each be practiced as extensions of the same disclosed framework rather than as departures from it.

The disclosed DNARP framework may be validated and used through computational, numerical, experimental, or combined validation pathways without requiring that any one validation pathway be treated as a limitation of the invention. In one embodiment, validation is performed computationally by executing the disclosed DNARP evaluation method on one or more candidate DNA programs and confirming that the system produces deterministic pass/fail outcomes, stability determinations, coherence-state determinations, and predicted functional outputs in accordance with the disclosed tuple structure, parameter relationships, governing constructs, and decision rules. In one embodiment, validation includes numerical solution or approximation of the disclosed structural stability and coherence formulations, together with computation of the disclosed transform-based output values for candidate programs representing therapeutic constructs, regulatory elements, DNA logic constructs, or other candidate DNA designs. In one embodiment, validation includes benchmarking the disclosed predicted outputs against known or measured expression-related, regulatory, switching, or other functional datasets so that predicted values may be compared to observed behavior across selected candidate classes.

In one embodiment, validation further includes experimental confirmation in which one or more candidate constructs identified by the disclosed DNARP framework are synthesized, assembled, or otherwise prepared and then assayed to compare predicted functional output, predicted enhancement, predicted switching behavior, or predicted target satisfaction against measured results. Such assays may include expression assays, reporter assays, regulatory-activity assays, switching assays, binding-associated assays, or other assay formats appropriate to the candidate type being evaluated. In each case, the practical utility of the invention resides in providing a formal, machine-implementable framework for representing candidate DNA designs, applying objective admissibility criteria to those designs, computing predicted functional outputs from validated candidate states, and using those results to select, reject, rank, refine, or further investigate candidate DNA programs in synthetic biology, therapeutic design, regulatory engineering, DNA computing, and related nucleic-acid design contexts. The foregoing validation pathways are exemplary means by which the disclosed framework may be confirmed, benchmarked, or applied in practice, and are not required to be performed in any particular order or combination unless expressly stated in a given embodiment.

What is claimed is:

1. A computer-implemented method for evaluating a candidate DNA design, the method comprising:
receiving, by one or more processors, a candidate DNA program comprising a sequence component, a shape component, and an energy component;
computing, from the sequence component, a sequence stability score for the candidate DNA program;
determining whether the shape component satisfies a helical pitch constraint and a groove-ratio constraint;
performing a structural stability evaluation for the candidate DNA program using a stability model that is based at least in part on a helical pitch of the shape component;
performing a coherence validation for the candidate DNA program by applying a DNA recognition operator to determine whether a coherence-related state of the energy component corresponds to an allowed discrete coherence state set under an operator-based validation framework;
computing, for the candidate DNA program, a predicted functional output based on the sequence stability score and the coherence-related state; and
outputting a result indicating validity or invalidity of the candidate DNA program and the predicted functional output.

2. The method of claim 1, wherein the candidate DNA program is represented as a tuple D=(S, H, E), where S is the sequence component, H is the shape component, and E is the energy component.

3. The method of claim 1, wherein computing the sequence stability score comprises computing the sequence stability score according to weighted CG and AT contributions.

4. The method of claim 3, wherein computing the sequence stability score comprises computing S_stab=1.5*(number of CG pairs)+1.0*(number of AT pairs), wherein the number of CG pairs comprises a number of Watson-Crick C·G base pairs and the number of AT pairs comprises a number of Watson-Crick A·T base pairs in a predicted paired region of the candidate DNA program.

5. The method of claim 1, wherein determining whether the shape component satisfies the helical pitch constraint and the groove-ratio constraint comprises determining whether a helical pitch P is within [30, 40] angstrom and whether a groove ratio G is within [1.5, 1.7].

6. The method of claim 1, wherein performing the structural stability evaluation comprises computing or approximating a structural profile C(r) using a DNA Lagrangian and determining whether abs (C(r)−1)<=0.05 over an evaluation domain.

7. The method of claim 1, wherein performing the coherence validation comprises determining whether an energy level E_n of the energy component corresponds to an allowed discrete state satisfying E_n=n*E_coh, where n is selected from {1, 2, 3}.

8. The method of claim 7, wherein the energy component further comprises an output rate R satisfying R=n*R_0.

9. The method of claim 1, wherein computing the predicted functional output comprises computing a recognition-transform-based output according to Output (D)=abs(F_DNA(E_n))\hat{ }2*R.

10. The method of claim 1, further comprising rejecting the candidate DNA program when the sequence stability score fails to satisfy a threshold criterion for at least one functional unit.

11. A system for evaluating a candidate DNA design, the system comprising:
one or more processors;
memory storing instructions executable by the one or more processors; and
one or more interfaces configured to receive a candidate DNA program,
wherein execution of the instructions causes the system to:
receive the candidate DNA program comprising a sequence component, a shape component, and an energy component;
compute a sequence stability score from the sequence component;
validate the shape component against a helical pitch constraint and a groove-ratio constraint;
evaluate structural stability of the candidate DNA program using a stability model based at least in part on a helical pitch of the shape component;
validate a coherence-related state of the energy component by applying a DNA recognition operator to determine whether the coherence-related state corresponds to an allowed discrete coherence state set under an operator-based validation framework;
compute a predicted functional output for the candidate DNA program; and
output a validity result and the predicted functional output.

12. The system of claim 11, wherein the one or more interfaces comprise an application programming interface configured to receive the candidate DNA program from an external software process.

13. The system of claim 11, further comprising non-transitory storage configured to store candidate DNA programs, predicted functional outputs, and candidate validity results.

14. The system of claim 11, wherein the instructions are further configured to cause the system to reject the candidate DNA program when the sequence stability score fails to satisfy a threshold criterion.

15. The system of claim 11, wherein the instructions are further configured to cause the system to reject the candidate DNA program when a requested coherence-related state does not correspond to an allowed discrete coherence state satisfying E_n=n*E_coh, where n is selected from {1, 2, 3}.

16. The system of claim 11, wherein the one or more interfaces and the one or more processors are further configured to support iterative refinement of the candidate DNA program by modifying at least the sequence component and re-evaluating the candidate DNA program.

17. A non-transitory computer-readable medium storing instructions that, when executed by one or more processors, cause the one or more processors to perform operations comprising:
receiving a candidate DNA program comprising a sequence component, a shape component, and an energy component;
computing a sequence stability score for the candidate DNA program;
determining whether the shape component satisfies a helical pitch constraint and a groove-ratio constraint;
performing a structural stability evaluation for the candidate DNA program using a stability model that is based at least in part on a helical pitch of the shape component;
performing a coherence validation for the candidate DNA program by applying a DNA recognition operator to determine whether a coherence-related state of the energy component corresponds to an allowed discrete coherence state set under an operator-based validation framework;
computing a predicted functional output for the candidate DNA program based on the sequence stability score and the coherence-related state; and
outputting a result indicating validity or invalidity of the candidate DNA program and the predicted functional output.

18. The non-transitory computer-readable medium of claim 17, wherein the operations further comprise iteratively modifying the sequence component and re-performing the computing, determining, performing, and outputting operations until a target output criterion is satisfied or a termination condition is reached.

19. The non-transitory computer-readable medium of claim 17, wherein the candidate DNA program corresponds to at least one of a therapeutic DNA construct, a promoter or regulatory element, or a DNA logic construct.

20. The non-transitory computer-readable medium of claim 17, wherein the operations further comprise rejecting the candidate DNA program when the sequence stability score fails to satisfy a threshold criterion for at least one functional unit.
